(19) United States
(12) Patent Application Publication
(10) Pub. No.: US 2023/0392119 A1
(43) Pub. Date: Dec. 7, 2023

(54) COMPOSITIONS AND METHODS FOR PREPARING ENGINEERED LYMPHOCYTES FOR CELL THERAPY

(71) Applicant: Kite Pharma, Inc., Foster City, CA (US)

(72) Inventors: Waleed Haso, Santa Monica, CA (US); Qi Cai, Champaign, IL (US); Thanh Nguyen Yip, San Francisco, CA (US); Hsing-Chuan Tsai, Santa Monica, CA (US); Carmen Warren, Los Angeles, CA (US); Melody Geragosian, Glendale, CA (US)

(21) Appl. No.: 18/324,012

(22) Filed: May 25, 2023

Related U.S. Application Data
(60) Provisional application No. 63/490,162, filed on Mar. 14, 2023.
(60) Provisional application No. 63/485,623, filed on Feb. 17, 2023.
(60) Provisional application No. 63/346,709, filed on May 27, 2022.

(51) Int. Cl. C12N 5/0783; A61K 39/00; A61P 35/00

(52) U.S. Cl. CPC C12N 5/0636; A61K 39/4611; A61K 39/4631; A61K 39/464412; A61K 39/464424; A61K 39/464466; A61P 35/00; C12N 2510/00; C12N 2533/52; A61K 2239/22

(57) ABSTRACT

Provided herein are compositions and methods for manufacturing engineered lymphocytes. Also provided are the prepared engineered lymphocytes which have increased proportions of juvenile/naive lymphocytes leading to increased therapeutic efficacy. The methods in various embodiments are expedited as compared to the conventional technology, and produce lymphocytes with improved viability, transduction success rates, and in vivo antitumor efficacy.

### CROSS-REFERENCE TO RELATED APPLICATIONS

The present application claims the priority benefit of U.S. Provisional Application No. 63/346,709, filed May 27, 2022, of U.S. Provisional Application No. 63/485,623, filed Feb. 17, 2023, and of U.S. Provisional Application No. 63/490,162, filed Mar. 14, 2023, which are hereby incorporated by reference in their entireties.

### TECHNICAL FIELD

The present disclosure relates to the field of cell therapy, and more specifically, compositions and methods for manufacturing engineered lymphocytes.

### BACKGROUND

Immune cells can be modified to target and kill cancer cells in a patient. To increase the ability of immune cells to target and kill a particular cancer cell, methods have been developed to engineer immune cells to express constructs which direct the immune cells to a particular target cancer cell. Chimeric antigen receptors (CARs) and engineered T cell receptors (TCRs), which include binding domains capable of interacting with a particular tumor antigen, allow the immune cells to target and kill cancer cells that express the particular tumor antigen.

A significant challenge comes with the highly complex autologous cell engineering and production process, which typically takes at least a week, and can take as long as several weeks. During the process, lymphocytes collected from the patients must be shipped to the process center, while the produced cells have to be cryopreserved and then shipped back to the patient for implantation. This highly complicated process necessarily leads to high costs, and limited clinical applications.

In addition, the lengthy process can result in reduced cell viability and increased lymphocyte maturity, both of which are detrimental to in vivo efficacy. There is a strong unmet need, therefore, to develop processes that not only are shorter in duration, but generate immune cells with improved therapeutic efficacy.

### SUMMARY

As provided, the current autologous CAR cell manufacturing process typically takes about 7 days and can be much longer. The instant disclosure describes improved processes that can be completed within 6 or even 4 days (or within 5 or even 3 days following an enrichment step). In various examples, the 5-day process (i.e., 5 days following enrichment) includes transduction preparation and implementation steps with a higher number of lymphocytes in contact with vectors immobilized to recombinant fibronectin coated to the inner surface of a closed system. Such an improved transduction procedure allows a much abbreviated post-transduction cell expansion step.

Yet a further improved process, which does not require post-transduction expansion at all, can be completed within only 3 days following an initial enrichment step. It was discovered that, surprisingly, both the 3-day and the 5-day processes produced transduced lymphocytes with higher percentages of juvenile cells. At least in part due to the increased juvenile cell population, these cell products exhibited greatly improved in vivo antitumor efficacy as compared to the conventional 7-day process.

In accordance with one embodiment of the present disclosure, therefore, provided is a method for preparing transduced lymphocytes, comprising incubating a sample comprising lymphocytes, obtained from a donor subject, with a polynucleotide vector to transduce the lymphocytes to produce transduced lymphocytes; and culturing the sample comprising the transduced lymphocytes for less than 72 hours before the lymphocytes are harvested to produce a harvested sample.

In some embodiments, the transduced lymphocytes are cultured for less than 48 hours before being harvested. In some embodiments, the transduced lymphocytes are cultured for less than 36 hours before being harvested.

In some embodiments, the incubation is carried out in a closed system. In some embodiments, the closed system has an inner surface area of at least 1500 cm². In some embodiments, the closed system has an inner surface coated with a recombinant human fibronectin, wherein the coating is carried out with a solution that comprises about 1-10 μg/ml of the recombinant human fibronectin. In some embodiments, the inner surface is further in contact with a second solution comprising the polynucleotide vector, wherein the second solution has a volume of about 200 mL. In some embodiments, the coating further comprises a drain of the second solution. In some embodiments, the sample in the closed system comprises at least 1.5×10⁸ lymphocytes. In some embodiments, the sample comprises at least 4×10⁸ lymphocytes.

In some embodiments, the lymphocytes are peripheral blood mononuclear cells (PBMCs) or T cells. In some embodiments, the harvested sample comprises CD3+ cells. In some embodiments, the harvested sample comprises CD4+ and CD8+ T cells. In some embodiments, at least 20% of the harvested CD4+ T cells are naïve T cells, and no more than 12% of the harvested CD4+ T cells are effector memory T cells. In some embodiments, at least 25% of the harvested CD4+ T cells are naïve T cells, and no more than 9% of the harvested CD4+ T cells are effector memory T cells. In some embodiments, at least 80% of the harvested CD4+ T cells are CCR7+ cells. In some embodiments, at most 20% of the harvested CD4+ T cells are a combination of effector memory T cells and effector T cells.

In some embodiments, at least 10% of the harvested CD8+ T cells are naïve T cells, and no more than 30% of the harvested CD8+ T cells are effector memory T cells. In some embodiments, at least 20% of the harvested CD8+ T cells are naïve T cells, and no more than 20% of the harvested CD8+ T cells are effector memory T cells. In some embodiments, the naïve T cells are characterized as CCR7+ and CD45RA+. In some embodiments, the effector memory T cells are characterized as CCR7−, CD45RO+ and CD95+. In some embodiments, at least 60% of the harvested CD8+ T cells are CCR7+ cells. In some embodiments, at most 40% of the harvested CD8+ T cells are a combination of effector memory T cells and effector T cells.

In some embodiments, the method further comprises acquiring the lymphocytes from the donor subject. In some embodiments, the method further comprises enriching the lymphocytes. In some embodiments, the method further comprises contacting the sample with a lymphocyte stimulating agent to activate the lymphocytes.

In some embodiments, the activating the sample is prior to incubating the sample with the polynucleotide vector. In some embodiments, the sample comprises at least 1×10⁹ lymphocytes. In some embodiments, the activating the sample is after the sample is incubated with the polynucleotide vector. In some embodiments, the lymphocyte stimulating agent comprises an anti-CD3 antibody and/or an anti-CD28 antibody.

In some embodiments, the method further comprises, following the harvesting, administering the harvested lymphocytes to a subject or freezing the harvested lymphocytes. In some embodiments, the subject is the same as the donor subject. In some embodiments, a total of to 3,000,000 harvested lymphocytes per kilogram of the subject are administered to the subject. In some embodiments, a total of 20,000 to 400,000 harvested lymphocytes per kilogram of the subject are administered to the subject. In some embodiments, at least 15% of the harvested lymphocytes are transduced with the vector.

In some embodiments, the polynucleotide vector is a viral vector. In some embodiments, the viral vector is a retroviral vector or a lentiviral vector. In some embodiments, the vector encodes a chimeric antigen receptor (CAR) or a T cell receptor (TCR). In some embodiments, the CAR comprises an intracellular costimulatory domain. In some embodiments, the intracellular costimulatory domain is a signaling region of a protein selected from the group consisting of DAP-CD28, OX-40, 4-1BB (CD137), CD2, CD7, CD27, CD30, CD40, programmed death-1 (PD-1), inducible T cell costimulator (ICOS), lymphocyte function-associated antigen-1 (LFA-1, CD11a/CD18), CD3 gamma, CD3 delta, CD3 epsilon, CD247, CD276 (B7-H3), tumor necrosis factor superfamily member 14, TNFSF14, LIGHT), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, IA4, CD49D, ITGA6, VLA-6, CD49f, ITGAD (CD11d), ITGAE (CD103), ITGAL (CD11a), ITGAM (CD11b), ITGAX (CD11c), ITGB1, CD29, ITGB2, CD18, ITGB7, NKG2D, TNFR2, TRANCE (RANKL), DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG (Cbp), CD19a, a ligand that specifically binds with CD83, and combinations thereof. In some embodiments, the intracellular costimulatory domain is a signaling region of CD28.

In some embodiments, the CAR or TCR recognizes a tumor antigen. In some embodiments, the tumor antigen is CD19. In some embodiments, the lymphocyte that comprises the CAR is axicabtagene ciloleucel or brexucabtagene autoleucel. In some embodiments, the tumor antigen is CD19 and/or CD20. In some embodiments, the tumor antigen is CLL-1.

In some embodiments, the CAR or TCR recognizes an antigen comprising 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), (32-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-O-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DRS, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRLS), fetal acetylcholine receptor (AChR), fibronectin extra domain-B, Fms-Like Tyrosine Kinase 3 (FLT3), folate-binding protein (FBP), folate receptor 1, folate receptor a, Folate receptor (3, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDG1cp(1-1)Cer), o-acetyl-GD2 ganglioside (OAcGD2), GITR (TNFRSF 18), GM1, ganglioside GM3 (aNeu5Ac(2-3)bDGalp(1-4)bDG1cp(1-1)Cer), GP 100, hexasaccharide portion of globoH glycoceramide (GloboH), glycoprotein 75, Glypican-3 (GPC3), glycoprotein 100 (gp100), GPNMB, G protein-coupled receptor 20 (GPR20), G protein-coupled receptor class C group 5, member D (GPRCSD), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAGS, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis (Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MART1, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRS S21), Proteinase3 (PRI), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1), or combinations thereof.

Also provided, in one embodiment, is a population of cells prepared from a blood sample of a donor subject, comprising CD4+ T cells and CD8+ T cells, wherein: at least 20% of harvested the CD4+ T cells are naïve T cells, and no more than 12% of the harvested CD4+ T cells are effector memory T cells; at least 10% of the harvested CD8+ T cells are naïve T cells, and no more than 30% of the harvested CD8+ T cells are effector memory T cells; at least 50% of the harvested cells are CD3+ T cells; and at least 25% of all T cells are transduced with a polynucleotide vector encoding a CAR or TCR.

Also provided, in one embodiment, is a population of cells prepared from a blood sample of a donor subject, comprising CD4+ T cells and CD8+ T cells, wherein: at least 80% of the CD4+ T cells are CCR7+ cells; at least 60% of the CD8+ T cells are CCR7+ cells; at most 20% of the CD4+ T cells are a combination of effector memory T cells and effector T cells; and at most 40% of the CD8+ T cells are a combination of effector memory T cells and effector T cells are transduced with a polynucleotide vector encoding a CAR or TCR.

In some embodiments, at least 25% of the harvested CD4+ T cells are naïve T cells, and no more than 9% of the harvested CD4+ T cells are effector memory T cells. In some embodiments, at least 20% of the harvested CD8+ T cells are naïve T cells, and no more than 20% of the harvested CD8+ T cells are effector memory T cells. In some embodiments, the naïve T cells are characterized as CCR7+ and CD45RA+. In some embodiments, the effector memory T cells are characterized as CCR7−, CD45RO+ and CD95+. In some embodiments, at least 65% of the harvested cells are CD3+ T cells. In some embodiments, at least 15% of all T cells are transduced with the polynucleotide vector.

In some embodiments, at least 80% of the harvested CD4+ T cells are CCR7+ cells. In some embodiments, at most 20% of the harvested CD4+ T cells are a combination of effector memory T cells and effector T cells. In some embodiments, at least 60% of the harvested CD8+ T cells are CCR7+ cells. In some embodiments, at most 40% of the harvested CD8+ T cells are a combination of effector memory T cells and effector T cells.

In some embodiments, the polynucleotide vector is a viral vector. In some embodiments, the viral vector is a retroviral vector or a lentiviral vector. In some embodiments, the CAR comprises an intracellular costimulatory domain. In some embodiments, the intracellular costimulatory domain is a signaling region of CD28. In some embodiments, the CAR recognizes a tumor antigen. In some embodiments, the tumor antigen is CD19. In some embodiments, the population of cells that comprise the CAR are axicabtagene ciloleucel or brexucabtagene autoleucel.

Also provided is pharmaceutical composition comprising the population of cells as described herein.

Yet another embodiment provides a method for administering T cells to a subject, comprising injecting to the subject a harvested sample prepared by the method of the present disclosure, or the pharmaceutical composition. In some embodiments, the subject has cancer. In some embodiments, the cancer is a B cell malignancy. In some embodiments, the cancer is Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, Lymphoblastic lymphoma, acute myeloid leukemia, or multiple myeloma.

### DETAILED DESCRIPTION

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation, OPTIPREP™ separation, and/or apheresis. Additional methods of isolating immune cells for an immune cell therapy are disclosed in U.S. Patent Publication No. 2013/0287748, which is herein incorporated by reference in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign nucleic acid is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

The term “7-day process” refers to a CAR cell manufacturing process which takes about 7 days following initial enrichment and/or activation step(s). The 7-day process is at least 8 days in length from the initial enrichment and/or activation step(s) to a harvesting step, and can be between 8 to 11 days in total when including the enrichment and/or activation step(s). In some embodiments, there is no initial activation step, or no activation prior to transduction.

The term “5-day process” refers to a CAR cell manufacturing process which takes about 5 days following initial enrichment and/or activation step(s). The 5-day process is 6 days in length from the initial enrichment and/or activation step(s) to a harvesting step, and can be between 6 to 9 days in total when including the enrichment and/or activation step(s). In some embodiments, there is no initial activation step, or no activation prior to transduction.

The term “3-day process” refers to a CAR cell manufacturing process which takes about 3 days from initial enrichment and/or activation step(s). The 3-day process is about 4 days in length from the initial enrichment and/or activation step(s) to a harvesting step. The 3-day process does not include a cell expansion step comprising one or more days following a transduction step and preceding a harvesting step. In some embodiments, there is no initial activation step, or no activation prior to transduction.

In some embodiments, the 3-day process described herein is about 5 days in length from the initial enrichment and/or activation step(s) to a harvesting step. In some embodiments, the 3-day process is about 3 to 4 days in length or about 72 to 96 hours in length from the initial enrichment and/or activation step(s) to a harvesting step (e.g., about 72 hours, 74 hours, 76 hours, 78 hours, 80 hours, 82 hours, 84 hours, 86 hours, 88 hours, 90 hours, 92 hours, 94 hours, 96 hours in length). In some embodiments, the 3-day process is about 4 to 5 days in length or about 96 to about 120 hours in length from the initial enrichment and/or activation step(s) to a harvesting step (e.g., about 96 hours, 98 hours, 100 hours, 102 hours, 104 hours, 106 hours, 10⁸ hours, 110 hours, 112 hours, 114 hours, 116 hours, 118 hours, 120 hours in length). In some embodiments, the 3-day process is less than 5 days or 120 hours in length from the initial enrichment and/or activation step(s) to a harvesting step.

### Preparation of Engineered Lymphocytes

The conventional autologous CAR cell manufacturing process takes about 7 days and can be much longer. The lengthy process was believed to be required at least because of the limited supply of starting materials, i.e., lymphocytes obtained from an apheresis collection from a donor subject, the relatively low-efficiency transduction, and the need to expand the transduced cells. Non-limiting examples of CAR cell manufacturing processes are described in WO2015120096 and WO2016191755, each of which is incorporated herein in its entirety.

As demonstrated in the accompanying examples, however, such a lengthy process is neither required nor beneficial to the patients. As shown in Table 7, for instance, each washing step during the expansion process considerably reduced the viability of the transduced cells. Also, the several-day expansion process, while increasing the total number of transduced cells, reduced the percentages of more juvenile ones (e.g., naïve T cells, stem memory T cells, and central memory T cells) and increase the percentages of the more mature ones (e.g., effector memory T cells) (Tables 2 and 6). The more juvenile lymphocytes, however, were shown to be more efficacious than the more mature ones (Tables 5, 10 and 11).

The instant disclosure describes improved processes that can be completed within 5 or even 3 days, following an enrichment step. In various examples, the 5-day process includes transduction preparation and implementation steps with a higher number of lymphocytes in contact with vectors immobilized to recombinant fibronectin coated to the inner surface of a closed system. Such an improved transduction procedure allows a much abbreviated post-transduction cell expansion step. As shown in Table 2, transduced cells prepared from the 5-day process skewed towards more juvenile cells. In vivo antitumor efficacy results are shown in (Table 5).

Yet a further improved process, which does not require post-transduction expansion at all, can be completed within only 3 days, following an enrichment step. Surprisingly, compared to the 5-day process, this 3-day process generates a population of cells with a greater percentage of juvenile cells and decreased percentage of more mature, differentiated and activated, cells (compared Table 6 to Table 2). Accordingly, not only the cell products from the 3-day process exhibited the best in vivo antitumor efficacy, such greatly improved efficacy could even be achieved with much lower doses (Tables 10-11).

In yet another surprising discovery, while the expedited lymphocyte preparation process improved the efficacy of all tested CAR types, it was particularly more beneficial to CAR molecules with a CD28 co-stimulatory domain (as compared to ones with a 4-1BB co-stimulatory domain) (Table 13).

In accordance with one embodiment of the present disclosure, therefore, provided is a method for preparing transduced (or transfected, in likewise manner) lymphocytes. In some embodiments, the method entails incubating a sample of lymphocytes with a polynucleotide vector to transduce the lymphocytes to produce transduced lymphocytes and culturing the sample that contains the transduced lymphocytes before the lymphocytes are harvested to produce a harvested sample.

In some embodiments, the culturing step is shortened as compared to the conventional process which takes about 4 days. In some embodiment, the culturing step is completed within 96 hours, or within 72 hours, 60 hours, 50 hours, 48 hours, 42 hours, 36 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, or 4 hours.

In some embodiments, the time for the culturing step is counted from completion of the transduction step (e.g., removal of the cells from the system with immobilized vectors) to harvesting of the cells for storage, transport, or clinical use.

Culturing of transduced lymphocytes can be done in media and conditions known in the art. In some embodiments, the culturing of the transduced lymphocytes may be performed at a temperature and/or in the presence of CO₂. In certain embodiments, the temperature may be about 34° C., about 35° C., about 36° C., about 37° C., about 38° C., or about 39° C. In certain embodiments, the temperature may be about 34-39° C. In certain embodiments, the predetermined temperature may be from about 35-37° C. In certain embodiments, the preferred predetermined temperature may be from about 36-38° C. In certain embodiments, the predetermined temperature may be about 36-37° C. or more preferably about 37° C.

In some embodiments, culturing of the transduced lymphocytes may be performed in the presence of a predetermined level of CO₂. In certain embodiments, the predetermined level of CO₂ may be 1.0-10% CO₂. In certain embodiments, the predetermined level of CO₂ may be about 1.0%, about 2.0%, about 3.0%, about 4.0%, about 5.0%, about 6.0%, about 7.0%, about 8.0%, about 9.0%, or about 10.0% CO₂. In certain embodiments, the predetermined level of CO₂ may be about 4.5-5.5% CO₂. In certain embodiments, the predetermined level of CO₂ may be about 5% CO₂. In certain embodiments, the predetermined level of CO₂ may be about 3.5%, about 4.0%, about 4.5%, about 5.0%, about 5.5%, or about 6.5% CO₂. In some embodiments, the step of expanding the population of transduced T cells may be performed at a predetermined temperature and/or in the presence of a predetermined level of CO₂ in any combination. For example, in one embodiment, the step of expanding the population of transduced T cells may comprise a predetermined temperature of about 36-38° C. and in the presence of a predetermined level of CO₂ of about 4.5-5.5% CO₂.

Any suitable culture medium T cell growth media may be used for culturing the cells in suspension. For example, a T cell growth media may include, but is not limited to, a sterile, low glucose solution that includes a suitable amount of buffer, magnesium, calcium, sodium pyruvate, and sodium bicarbonate. In one embodiment, the culturing media is OpTmizer™ (Life Technologies), but one skilled in the art would understand how to generate similar media. In one embodiment, the culturing media is EX-VIVO™ serum free media (Lonza Bioscience).

The incubation (and/or transduction) step can be carried out in a closed system, without limitation. In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags). In some embodiments, the closed system has an inner surface area of at least 500 cm². In some embodiments, the closed system has an inner surface area of at least 1000 cm², 1200 cm², 1400 cm², 1500 cm², 1600 cm², 1800 cm², 2000 cm², 2200 cm², 2500 cm², or 3000 cm². In some embodiments, the closed system has an inner surface area of not greater than 1500 cm², 1600 cm², 1800 cm², 2000 cm², 2200 cm², 2500 cm², or 3000 cm².

In some embodiments, the cell culture bags used in the closed system are coated with a recombinant human fibronectin protein. The recombinant human fibronectin fragment may include three functional domains: a central cell-binding domain, heparin-binding domain II, and a CS1-sequence. The recombinant human fibronectin protein or fragment thereof may be used to increase gene efficiency of viral transduction of immune cells by aiding co-localization of target cells or the vector. In certain embodiments, the recombinant human fibronectin fragment is RetroNectin® (Takara Bio, Japan). In certain embodiments, the cell culture bags may be coated with recombinant human fibronectin fragment at a concentration of about 0.1-60 μg/mL, preferably 0.5-40 μg/mL. In certain embodiments, the cell culture bags may be coated with recombinant human fibronectin fragment at a concentration of about 0.5-20 μg/mL, 20-40 μg/mL, or 40-60 μg/mL. In certain embodiments, the cell culture bags may be coated with about μg/mL, 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In certain embodiments, the cell culture bags may be coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags may be coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In certain embodiments, the cell culture bags may be coated with at least about 10 μg/mL recombinant human fibronectin fragment. In certain embodiments, the cell culture bags may not be coated with recombinant human fibronectin fragment.

In some embodiments, a transduction enhancing agent is introduced into the closed system. Non-limiting examples of such transduction enhancing agents include Vectofusin™ transduction mixtures.

In certain embodiments, the cell culture bags used in the closed bag culture system may be blocked with human albumin serum (HSA). In an alternative embodiment, the cell culture bags are not blocked with HSA.

Once the closed system is coated with the recombinant fibronectin, a solution that includes the vector is added to the closed system so that the vector can be immobilized by the recombinant fibronectin, on the inner surface of the closed system. Such immobilization can improve the transduction efficiency once the cells are added.

In some embodiments, the vectors can be viral vectors, such as lentiviral vectors, as well as retroviral vectors. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. In one embodiment, the viral vector is an MSGV1 gamma retroviral vector. In some embodiments, the vectors are non-viral vectors.

In some embodiments, a total volume of at least 100 mL of the solution that contains the vector is used. In some embodiments, a total volume of at least 110 mL, 120 mL, 130 mL, 140 mL, 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, or 400 mL of the solution that contains the vector is used. In some embodiments, a total volume of no more than 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, 400 mL, or 500 mL of the solution that contains the vector is used.

In some embodiments, the vector solution includes at between 1×10³ to 1×10¹² transduction units per milliliter (TU/ml) of the viral vector.

Once the closed system is coated with the recombinant fibronectin and has immobilized the vector, the vector solution can be removed. In some embodiments, the closed system does not include recombinant fibronectin. In some embodiments, the removal of the vector solution is done by gravity or syringe drain, which helps to retain the immobilized vector on the inner surface while removing impurities. Lymphocyte transduction can be carried in the coated closed system with the immobilized vectors. In some embodiments, the transduction is performed with a sample that contained the lymphocytes. In some embodiments, the sample includes at least 2.5×10⁷ lymphocytes (e.g., T cells). In some embodiments, the sample includes at least 3×10⁷, 4×10⁷, 5×10⁷, 6×10⁷, 7×10⁷, 8×10⁷, 9×10⁷, 1×10⁸, 1.2×10⁸, 1.5×10⁸, 1.8×10⁸, 2×10⁸, 2.2×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells). In some embodiments, the sample includes no more than 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells).

In some embodiments, lymphocyte transduction is carried out in a closed system that does not contain an immobilizing agent (e.g., recombinant fibronectin).

The lymphocytes used in the presently disclosed methods are typically obtained from a donor subject, which may be a cancer patient that is to be treated with a population of cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). The lymphocytes may be obtained from the donor subject by any suitable method used in the art. For example, the lymphocytes may be obtained by any suitable extracorporeal method, venipuncture, or other blood collection method by which a sample of blood and/or lymphocytes is obtained. In one embodiment, the lymphocytes are obtained by apheresis.

Optionally, in some embodiments, the method described herein further includes a step of enriching a population of lymphocytes obtained from the donor subject, prior to the transduction. Enrichment of lymphocytes may be accomplished by any suitable separation method including, but not limited to, the use of a separation medium (e.g., Ficoll-Paque™, RosetteSep™ HLA Total Lymphocyte enrichment cocktail, Lymphocyte Separation Medium (LSA) (MP Biomedical Cat. No. 0850494X), a non-ionic iodixanol-based medium such as OptiPrep™, or the like), cell size, shape or density separation by filtration or elutriation, immunomagnetic separation (e.g., magnetic-activated cell sorting system, MACS), fluorescent separation (e.g., fluorescence activated cell sorting system, FACS), or bead based column separation.

Optionally, in some embodiments, circulating lymphoma cells are removed from the sample through positive enrichment for CD4⁺/CD8⁺ cells via the use of selection reagents. In some such embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some such embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps may be based on positive selection, in which the cells having bound the reagents are retained for further use, and/or negative selection, in which the cells having not bound to the antibody or binding partner are retained. In some examples, both fractions are retained for further use.

In some such embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+, CD28+ T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+ or CD8+selection step is used to separate CD4+ helper and CD8+ cytotoxic T cells. Such CD4+ and CD8+ populations may be further sorted into subpopulations by positive or negative selection for markers expressed or expressed to a relatively higher degree on one or more naive, memory, and/or effector T cell subpopulations.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques. In some embodiments, the sample or composition of cells to be separated is incubated with small, magnetizable or magnetically responsive material, such as magnetically responsive particles or microparticles, such as paramagnetic beads (e.g., such as Dynabeads™ or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample. In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps. In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies. In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 A1, which are each incorporated herein by reference. In some embodiments, the system or apparatus carries out one or more, e.g., of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps. In some embodiments, the separation and/or other steps is carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some embodiments is equipped with a cell processing unity that permits automated washing and fractionation of cells by centrifugation. The CliniMACS Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, at least 0.5×10⁹ lymphocytes are acquired from the donor, and are optionally enriched and/or subjected to the stimulation. In some embodiments, at least 0.6×10 9, 0.7×10⁹, 0.8×10⁹, 0.9×10⁹, 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation. In some embodiments, no more than 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation.

Also optionally, the methods described herein further includes a step of stimulating the lymphocytes with one or more lymphocyte stimulating agents. In some embodiments, the stimulation is performed prior to the transduction step. In some embodiments, the stimulation is performed after the transduction step. The stimulation step is also referred to herein as an activation step.

Any combination of one or more suitable lymphocyte stimulating agents may be used to stimulate (activate) the lymphocytes. Non-limiting examples include an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. In some embodiments, the stimulating agent is an anti-CD3 antibody and/or an anti-CD28 antibody.

In some embodiments, the lymphocyte stimulating agent may be a bead-based activator, such as T-cell TransAct™ (Miltenyi Biotec), Dynabeads® (Thermo Fisher Scientific), or Cloudz™ T Cell Activation Kit (R&D Systems).

In some embodiments, the step of stimulating lymphocytes as described herein may entail stimulating the lymphocytes with one or more stimulating agents at a predetermined temperature, for a predetermined amount of time, and/or in the presence of a predetermined level of CO₂. In certain embodiments, the predetermined temperature for stimulation may be about 34° C., about 35° C., about 36° C., about 37° C., about 38° C., or about 39° C. In certain embodiments, the predetermined temperature for stimulation may be about 34-39° C. In certain embodiments, the step of stimulating the lymphocytes comprises stimulating the lymphocytes with one or more stimulating agents for a predetermined time. In certain embodiments, the predetermined time for stimulation may be about 24-72 hours. In certain embodiments, the predetermined time for stimulation may be about 24-36 hours. In certain embodiments, the step of stimulating the lymphocytes may comprise stimulating the lymphocytes with one or more stimulating agents in the presence of a predetermined level of CO₂. In certain embodiments, the predetermined level of CO₂ for stimulation may be about 1.0-10% CO₂. In certain embodiments, the predetermined level of CO₂ for stimulation may be about 1.0%, about 2.0%, about 3.0%, about 4.0%, about 5.0%, about 6.0%, about 7.0%, about 8.0%, about 9.0%, or about 10.0% CO₂.

In some embodiments, an anti-CD3 antibody (or functional fragment thereof), an anti-CD28 antibody (or functional fragment thereof), or a combination of anti-CD3 and anti-CD28 antibodies may be used in accordance with the step of stimulating the population of lymphocytes. Any soluble or immobilized anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. Any antibody used in the methods described herein should be produced under Good Manufacturing Practices (GMP) to conform to relevant agency guidelines for biologic products.

In certain embodiments, the T cell stimulating agent may include an anti-CD3 or anti-CD28 antibody at a concentration of from about 20 ng/mL-100 ng/mL. In certain embodiments, the concentration of anti-CD3 or anti-CD28 antibody may be about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL.

In some embodiments, a 3-day process of the present disclosure may comprise the following steps: apheresis material collection, a first wash step conducted on day 0, a second wash step conducted on day 0, an enrichment step conducted on day 0, a third wash step conducted on day 0, an activation step conducted on day 0 up to day 1, a viral transduction step conducted on day 1 up to day 4, and a fourth wash and concentration step conducted on day 3 up to day 4 (the day of harvest). The apheresis material may be fresh apheresis material, cryopreserved apheresis material, or cryopreserved T cells.

In some embodiments, the 3-day process may comprise an activation step on day 0, day 1, or day 2. In some embodiments, the 3-day process may comprise a viral vector transduction step on day 1, day 2, or day 3.

In some embodiments, the 3-day process may optionally comprise one, two, three, four, five, six, seven, eight or more wash steps. Each wash step may comprise the same wash procedure or a different wash procedure. In some embodiments, one or more wash steps may occur prior to the enrichment step. In some embodiments, the first wash may occur after the enrichment step. In some embodiments, the first wash may occur after the enrichment step and before the activation step. In some embodiments, the first wash may occur after both the enrichment and activation steps. In some embodiments, a wash step may be conducted on the day of harvest.

### Prepared Engineered Lymphocytes

The lymphocytes prepared, as demonstrated in the accompanying experimental examples, included higher ratios of juvenile ones (e.g., naïve T cells). Accordingly, one embodiment of the present disclosure provides a population of lymphocytes, prepared by the instant methods, that include CD4+ and CD8+ T cells.

In some embodiments, at least 20% of the CD4+ T cells are naïve T cells. In some embodiments, at least 25%, 30%, 35%, 40%, 45%, 50%, 55% or 60% of the CD4+ T cells are naïve T cells.

In some embodiments, no more than 25% of the CD4+ T cells are effector memory T cells. In some embodiments, no more than 20%, 19%, 18%, 17%, 16%, 15%, 14%, 13%, 12%, 11%, 10%, 9%, 8%, 7%, 6% or 5% of the CD4+ T cells are effector memory T cells.

In some embodiments, no more than 44% of the CD4+ T cells are central memory T cells. In some embodiments, no more than 43%, 42%, 41%, or 40% of the CD4+ T cells are central memory T cells.

In some embodiments, no more than 1.5% of the CD4+ T cells are effector T cells. In some embodiments, no more than 1.4%, 1.3%, 1.2%, 1.1%, 1%, 0.9%, 0.8%, 0.7%, 0.6% or 0.5% of the CD4+ T cells are effector T cells.

In some embodiments, at least 80% of the harvested CD4+ T cells are CCR7+ cells. In some embodiments, at most 20% of the harvested CD4+ T cells are a combination of effector memory T cells and effector T cells.

In some embodiments, at least 5% of the CD8+ T cells are naïve T cells. In some embodiments, at least 10%, 15%, 20%, 25%, 30% or 35% of the CD8+ T cells are naïve T cells.

In some embodiments, no more than 30% of the CD8+ T cells are effector memory T cells. In some embodiments, no more than 28%, 27%, 25%, 22%, 20%, 19%, 18%, 17%, 16%, 15%, 14%, 13%, 12%, 11%, or 10% of the CD8+ T cells are effector memory T cells.

In some embodiments, no more than 60% of the CD8+ T cells are central memory T cells. In some embodiments, no more than 58%, 56%, 55%, 54%, 52% or 50% of the CD8+ T cells are central memory T cells.

In some embodiments, at least 60% of the harvested CD8+ T cells are CCR7+ cells. In some embodiments, at most 40% of the harvested CD8+ T cells are a combination of effector memory T cells and effector T cells.

Each type of T cells can be characterized with cell surface markers, as well known in the art. For instance, naïve T cells can be characterized as CCR7+, CD45RO−, and CD95−. Additional markers for naïve T cell include CD45RA+, CD62L+, CD27+, CD28+, CD127+, CD132+, CD25−, CD44−, and HLA-DR−.

Surface markers to stem memory T cells (Tscm) include, without limitation, CD45RO−, CCR7+, CD45RA+, CD62L+(L-selectin), CD27+, CD28+, IL-7Ra+, CD95+, IL-2Rβ+, CXCR3+, and LFA−.

Surface markers for effector memory T cells (Tem) include, without limitation, CCR7−, CD45RO+ and CD95+. Additional marker for effector memory T cells is IL-2Rβ+. For central memory T cells (Tcm), suitable markers include CD45RO+, CD95+, IL-2Rβ+, CCR7+ and CD62L+. For effector T cells (Teff), suitable markers include CD45RA+, CD95+, IL-2Rβ+, CCR7− and CD62L−, without limitation.

The term “juvenile cells” as referred throughout includes one or more of naïve T cells, stem memory T cells (Tscm), and central memory T cells (Tcm). These cells are characterized, in part, by expression of CCR7+.

The harvested lymphocytes preferably include a good proportion that is CD3+ T cells. In some embodiments, at least 25%, 35%, 45%, 50%, 55%, 60%, 65%, 70%, 75%, 80%, 85% or 90% of the harvested lymphocytes are CD3+ T cells.

The harvested lymphocytes preferably include a good proportion that has been transduced. In some embodiments, at least 5%, 6%, 7%, 8%, 9%, 10%, 11%, 12%, 13%, 14% 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 44%, 45%, 46%, 47%, 48%, 49%, or 50% of the harvested lymphocytes are transduced with the vector. In some embodiments, each transduced lymphocyte includes at least a copy of the vector (or the included coding sequence) integrated to the host genome. In some embodiments, each transduced lymphocyte includes at least 2, 3, 4, 5, 6, 7, 8, 9 or 10 copies of the vector integrated to the host genome.

In some embodiments, the vector includes a transgene that encodes a polypeptide. The polypeptide, without limitation, may be a CAR or TCR. In some embodiments, the CAR or TCR includes an antigen binding molecule. The antigen binding molecule, in some embodiments, has binding specificity to an antigenic moiety. In some embodiments, the antigenic moiety is a tumor antigen (e.g., a protein or other molecule that is produced by a cancer cell).

In some embodiments, the vector includes more than one transgene that encodes for more than one CAR or TCR molecule that include antigen binding molecules with specificity to different antigenic moieties. In some embodiments, the vector includes more than one transgene that encodes for more than one CAR or TCR molecule that include antigen binding molecules with specificity to two different tumor antigens.

In some aspects, the antigenic moiety is an antigen associated with a cancer or a cancer cell. Such antigens may include, but are not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor—2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), L1CAM (L1 cell adhesion molecule), MAGE (melanoma antigen), MAGE-A1 (Melanoma-associated antigen 1), mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X 1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene), or combinations thereof.

Additional examples of tumor antigens include 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), 32-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B 1), DNAM-1 (CD226), desmoglein 4, DR3, DRS, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRLS), fetal acetylcholine receptor (AChR), fibronectin extra domain-B, Fms-Like Tyrosine Kinase 3 (FLT3), folate-binding protein (FBP), folate receptor 1, folate receptor a, Folate receptor (3, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDG1cp(1-1)Cer), o-acetyl-GD2 ganglioside (OAcGD2), GITR (TNFRSF 18), GM1, ganglioside GM3 (aNeu5Ac(2-3)bDGalp(1-4)bDG1cp(1-1)Cer), GP 100, hexasaccharide portion of globoH glycoceramide (GloboH), glycoprotein 75, Glypican-3 (GPC3), glycoprotein 100 (gp100), GPNMB, G protein-coupled receptor 20 (GPR20), G protein-coupled receptor class C group 5, member D (GPRCSD), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis (Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MART1, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PRI), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1).

In other embodiments, the antigenic moiety is associated with virally infected cells (i.e., a viral antigenic moiety). Such antigenic moieties may include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL, gM, UL20, UL32, US43, UL45, UL49A), a cytomegalovirus (CMV) viral antigen (e.g., glycoproteins gB, gC, gC, gE, gG, gH, gI, gJ, gK, gL, gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such embodiments, the cell surface receptor may be any TCR, or any CAR which recognizes any of the aforementioned viral antigens on a target virally infected cell.

In other embodiments, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

In some embodiments, the TCR has specificity to an antigenic moiety on cancer cells. Non-limiting examples of TCR include an anti-707-AP TCR, anti-AFP TCR, anti-ART-4 TCR, anti-BAGE TCR, anti-Bcr-abl TCR, anti-CAMEL TCR, anti-CAP-1 TCR, anti-CASP-8 TCR, anti-CDC27m TCR, anti-CDK4/m TCR, anti-CEA TCR, anti-CT TCR, anti-Cyp-B TCR, anti-DAM TCR, anti-TCR, anti-EGFRvIII TCR, anti-ELF2M TCR, anti-ETV6-AML1 TCR, anti-G250 TCR, GAGE TCR, anti-GnT-V TCR, anti-Gp100 TCR, anti-HAGE TCR, anti-HER-2/neu TCR, anti-HLA-A TCR, anti-HPV TCR, anti-HSP70-2M TCR, anti-HST-2 TCR, anti-hTERT TCR or anti-hTRT TCR, anti-iCE TCR, anti-KIAA0205, anti-LAGE (L antigen), anti-LDLR/FUT TCR, anti-MAGE TCR, anti-MART-1/Melan-A TCR, anti-MC1R TCR, anti-Myosin/m TCR, anti-MUC1 TCR, anti-MUM-1, -2, -3 TCR, anti-NA88-A TCR, anti-NY-ESO-1 TCR, anti-P15 TCR, anti-p190 minor bcr-abl TCR, anti-Pml/RARa TCR, anti-PRAME TCR, anti-PSA TCR, anti-PSMA TCR, anti-RAGE TCR, anti-RU1 TCR or anti-RU2 TCR, anti-SAGE TCR, anti-SART-1 TCR or anti-SART-3 TCR, anti-SSX1, -2, -3, 4 TCR, anti-TEL/AML1 TCR, anti-TPI/m TCR, anti-TRP-1 TCR, anti-TRP-2 TCR, anti-TRP-2/INT2 TCR, or anti-WT1 TCR.

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly10⁸), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAM1, CRT AM, cytokine receptor, DAP-DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-1BB costimulatory domain. In certain instances, a CAR includes a CD28 costimulatory domain. In certain instances, a CAR includes a DAP-10 costimulatory domain.

In some embodiments, the costimulatory signaling domain is a signaling domain of CD28. As shown in the experimental examples, CAR molecules with a CD28 costimulatory signaling domain can particularly benefit from the newly developed, expedited manufacturing process. In some embodiments, the CAR is encoded by a nucleic acid molecule embedded in a viral vector. In some embodiments, the CAR is encoded by a nucleic acid molecule embedded in a lentiviral vector.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d. In some embodiments, the ITAM includes CD3 zeta.

In some embodiments, the CAR molecule may be any anti-CD19 CAR molecule. In one aspect the anti-CD19 CAR includes an extracellular scFv domain, an intracellular and/or transmembrane, portion of a CD28 molecule, an optional extracellular portion of the CD28 molecule, and an intracellular CD3zeta domain as described in WO2015120096 or WO2016191755, each of which is incorporated herein in its entirety.

In certain embodiments, the anti-CD19 CAR may also include additional domains, such as a CD8 extracellular and/or transmembrane region, an extracellular immunoglobulin Fc domain (e.g., lgG1, lgG2, lgG3, lgG4), or one or more additional signaling domains, such as 41 BB, OX40, CD2 CD16, CD27, CD30 CD40, PD-1, ICOS, LFA-1, IL-2 Receptor, Fc gamma receptor, or any other costimulatory domains with immunoreceptor tyrosine-based activation motifs.

In certain embodiments, the cell surface receptor is an anti-CD19 CAR, such as FMC63-28Z CAR or FMC63-CD828BBZ CAR as set forth in Kochenderfer et al., J Immunother. 2009 September; 32(7): 689-702, “Construction and Preclinical Evaluation of an Anti-CD19 Chimeric Antigen Receptor,” the subject matter of which is hereby incorporated by reference for the purpose of providing the methods of constructing the vectors used to produce T cells expressing the FMC63-28Z CAR or FMC63-CD828BBZ CAR.

In some embodiments, the T cell that includes a CAR molecule is Yescarta® (axicabtagene ciloleucel). In some embodiments, the T cell that includes a CAR molecule is Tecartus® (brexucabtagene autoleucel).

In some embodiments, the engineered lymphocytes comprise a dual-targeted antigen binding system. Dual-targeted antigen binding systems may comprise bispecific CARs or TCRs and/or bicistronic CARs or TCRs. Bispecific and bicistronic CARs can comprise two binding motifs (in a single CAR molecule or in two CAR molecules, respectively). In some embodiments, the vector of the present disclosure encodes bicistronic and/or bispecific CARs (e.g., bicistronic and/or bispecific CARs that bind CD20 and CD19).

In some embodiments, a pharmaceutical composition is provided that includes a population of engineered lymphocytes produced by the methods described herein. In certain embodiments, the pharmaceutical composition may also include a pharmaceutically acceptable carrier. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

### Treatments and Uses, and Optional Storage

The lymphocytes prepared by the instant methods, or the lymphocyte populations as disclosed herein, can be used for treating various diseases and conditions.

In some embodiments, if the lymphocytes are not immediately used, they can cryopreserved so that they can be used at a later date. Such a method may include a step of washing and concentrating the population of engineered lymphocytes with a diluent solution. In some aspects the diluent solution is normal saline, 0.9% saline, PlasmaLyte A (PL), 5% dextrose/0.45% NaCl saline solution (D5), human serum albumin (HSA), or a combination thereof. In some aspects, HSA may be added to the washed and concentrated cells for improved cell viability and cell recovery after thawing. In another aspect, the washing solution is normal saline and washed and concentrated cells are supplemented with HSA (5%). The method may also include a step of generating a cryopreservation mixture, wherein the cryopreservation mixture includes the diluted population of cells in the diluent solution and a suitable cryopreservative solution. In some aspects, the cryopreservative solution may be any suitable cryopreservative solution including, but not limited to, CryoStor10 (BioLife Solution), mixed with the diluent solution of engineered lymphocytes at a ratio of 1:1 or 2:1.

In certain embodiments, HSA may be added to provide a final concentration of about 1.0-10% HSA in the cryopreserved mixture. In certain embodiments, HSA may be added to provide a final concentration of about 1.0%, about 2.0%, about 3.0%, about 4.0%, about 5.0%, about 6.0%, about 7.0%, about 8.0%, about 9.0%, or about 10.0% HSA in the cryopreserved mixture. In certain embodiments, HSA may be added to provide a final concentration of about 1-3% HSA, about 1-4% HSA, about 1-5% HSA, about 1-7% HSA, about 2-4% HSA, about 2-5% HSA, about 2-6% HSA, or about 2-7% HSA in the cryopreserved mixture. In certain embodiments, HSA may be added to provide a final concentration of about 2.5% HSA in the cryopreserved mixture. For example, in certain embodiments, cryopreservation of a population of engineered T cells may comprise washing cells with 0.9% normal saline, adding HSA at a final concentration of 5% to the washed cells, and diluting the cells 1:1 with CryoStor™ CS10 (for a final concentration of 2.5% HSA in the final cryopreservation mixture). In some embodiments, the method also includes a step of freezing the cryopreservation mixture. In one aspect, the cryopreservation mixture is frozen in a controlled rate freezer using a defined freeze cycle at a cell concentration of between about 1e6 to about 1.5e7 cells per mL of cryopreservation mixture. The method may also include a step of storing the cryopreservation mixture in vapor phase liquid nitrogen.

Methods and uses are also provided, for treating a disease or pathological condition in a subject having the disease or pathological condition. In some embodiments, the method entails administering a therapeutically effective amount or therapeutically effective dose of the engineered lymphocytes to the subject. Pathogenic conditions that may be treated with engineered T cells that are produced by the methods described herein include, but are not limited to, cancer, viral infection, acute or chronic inflammation, autoimmune disease or any other immune-dysfunction.

As referred to herein, a “cancer” may be any cancer that is associated with a surface antigen or cancer marker, including, but not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In some aspects, the cancer is a B cell malignancy. Examples of B cell malignancies include, but are not limited to, Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma and Lymphoblastic lymphoma. As referred to herein, a “viral infection” may be an infection caused by any virus which causes a disease or pathological condition in the host. Examples of viral infections that may be treated with the engineered T cells that are produced by the methods described herein include, but are not limited to, a viral infection caused by an Epstein-Barr virus (EBV); a viral infection caused by a hepatitis A virus, a hepatitis B virus or a hepatitis C virus; a viral infection caused by a herpes simplex type 1 virus, a herpes simplex type 2 virus, or a herpes simplex type 8 virus, a viral infection caused by a cytomegalovirus (CMV), a viral infection caused by a human immunodeficiency virus (HIV), a viral infection caused by an influenza virus, a viral infection caused by a measles or mumps virus, a viral infection caused by a human papillomavirus (HPV), a viral infection caused by a parainfluenza virus, a viral infection caused by a rubella virus, a viral infection caused by a respiratory syncytial virus (RSV), or a viral infection caused by a varicella-zostser virus. In some aspects, a viral infection may lead to or result in the development of cancer in a subject with the viral infection (e.g., HPV infection may cause or be associated with the development of several cancers, including cervical, vulvar, vaginal, penile, anal, oropharyngeal cancers, and HIV infection may cause the development of Kaposi's sarcoma). Examples of chronic inflammation diseases, autoimmune diseases or any other immune-dysfunctions that may be treated with the engineered T cells produced by the methods described herein include, but are not limited to, multiple sclerosis, lupus, and psoriasis.

Additional examples of chronic inflammation diseases, autoimmune diseases or any other immune-dysfunctions that may be treated with the engineered T cells produced by the methods described herein include rheumatoid arthritis, allergies, asthma, Crohn's disease, IBD, IBS, fibromyalgia, mastocytosis, and Celiac disease.

The term “treat,” “treating” or “treatment” as used herein with regard to a condition or disease may refer to preventing a condition or disease, slowing the onset or rate of development of the condition or disease, reducing the risk of developing the condition or disease, preventing or delaying the development of symptoms associated with the condition or disease, reducing or ending symptoms associated with the condition or disease, generating a complete or partial regression of the condition or disease, or some combination thereof.

A “therapeutically effective amount” or a “therapeutically effective dose” is an amount of engineered lymphocytes that produce a desired therapeutic effect in a subject, such as preventing or treating a target condition or alleviating symptoms associated with the condition by killing target cells. The most effective results in terms of efficacy of treatment in a given subject will vary depending upon a variety of factors, including but not limited to the characteristics of the engineered lymphocytes (including longevity, activity, pharmacokinetics, pharmacodynamics, and bioavailability), the physiological condition of the subject (including age, sex, disease type and stage, general physical condition, responsiveness to a given dosage, and type of medication), the nature of any pharmaceutically acceptable carrier or carriers in any composition used, and the route of administration. A therapeutically effective dose of engineered lymphocytes also depends on the cell surface receptor that is expressed by the lymphocytes (e.g., the affinity and density of the cell surface receptors expressed on the cell), the type of target cell, the nature of the disease or pathological condition being treated, or a combination of both.

As shown in the examples, the engineered lymphocytes prepared by the instant process have greatly increased in vivo efficacy and thus a much lower dose is required, as compared to the conventional technology.

Therefore, in some aspects, a therapeutically effective dose of engineered lymphocytes is fewer than about 2 million engineered lymphocytes per kilogram of body weight of the subject in need of treatment (cells/kg). Therefore, in some aspects, a therapeutically effective dose of engineered lymphocytes is from about 10,000 to about 2,500,000 engineered lymphocytes/kg. In certain embodiments, a therapeutically effective dose of engineered lymphocytes is from about to about 1,500,000 engineered lymphocytes/kg. In certain embodiments, the therapeutically effective dose is about 20,000 to about 1,200,000 engineered lymphocytes/kg. In certain embodiments, the therapeutically effective dose is about 20,000 to about 1,000,000 engineered lymphocytes/kg. In certain embodiments, the therapeutically effective dose is about to about 500,000 engineered lymphocytes/kg. In certain embodiments, the therapeutically effective dose is about 20,000 to about 400,000 engineered lymphocytes/kg. In certain embodiments, the therapeutically effective dose is about 40,000 to about 400,000 engineered lymphocytes/kg. In certain embodiments, the therapeutically effective dose is about 50,000 to about 200,000 engineered lymphocytes/kg. In certain embodiments, the therapeutically effective dose is about 50,000 to about 100,000 engineered lymphocytes/kg.

In some aspects, a therapeutically effective dose of engineered lymphocytes is from about 1,600,000 to about 2,500,000 engineered lymphocytes per kilogram of body weight of the subject in need of treatment (cells/kg). In some embodiments, the therapeutically effective dose of engineered lymphocytes is from about 2,000,000 to about 2,400,000 engineered lymphocytes per kilogram of body weight of the subject in need of treatment (cells/kg).

In some embodiments, the T cells administered are Yescarta® (axicabtagene ciloleucel). In some embodiments, the T cells administered are Tecartus® (brexucabtagene autoleucel).

The following examples are intended to illustrate various embodiments of the invention. As such, the specific embodiments discussed are not to be construed as limitations on the scope of the invention. For example, although the Examples below are directed to T cells transduced with an anti-CD19 chimeric antigen receptor (CAR), one skilled in the art would understand that the methods described herein may apply to T cells transduced with any CAR. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Seven Day Lymphocyte Manufacturing Process

This example describes a process of preparing lymphocytes that are transduced with a polynucleotide vector, such a viral vector, that encodes a therapeutic protein. The prepared lymphocytes can be useful for treating various diseases such as cancer, especially when the therapeutic protein is a chimeric antigen receptor (CAR) or T cell receptor (TCR) designed to target a cancer cell.

As used throughout, the terms “7-day process” and “7-day lymphocyte manufacturing process” are used interchangeably and refer to a CAR cell manufacturing process which takes about 7 days following initial enrichment and activation steps. The 7-day process is at least 8 days in length from the initial enrichment and activation steps to a harvesting step, and can be between 8 to 11 days in total when including the enrichment and activation steps.

Apheresis Collection. White blood cells were collected (leukapheresis) using standard apheresis equipment, such as Cobe® Spectra, Spectra Optia®, Fenwal™ Amicus® or equivalent. The leukapheresis process typically yielded approximately 200-400 mL of an apheresis product from patients. The apheresis product may be subjected to the manufacturing process on-site, or optionally shipped at 1-10° C. to a facility to undergo the manufacturing process in a different location. Further process steps may be conducted in an ISO 7 cell culture process suite (or similar clean room type environment).

Volume Reduction. Where appropriate, a volume reduction step was performed using a cell processing instrument such as the Sepax® 2 laboratory instrument (Biosafe SA; Houston, TX) or equivalent, and carried out using a standard aseptic tubing kit. Given the variability in the number of cells and volume of incoming source material from each subject (approximately 200-400 mL), the volume reduction step is designed to standardize the volume of cells to approximately 120 mL. In the event that the apheresis volume is less than 120 mL, the volume reduction step need not be performed, and the cells directly carried to the lymphocyte enrichment step. The volume reduction step is designed to standardize the volume of cells received from each subject, retain mononuclear cells, achieve consistent cell yield and high cell viability, and maintain a closed system to minimize risk of contamination.

Lymphocyte Enrichment. Following the Volume Reduction step, the cells were subjected to Ficoll based separation on a cell processing instrument, such as the Sepax® 2 or equivalent, using the separation protocol developed and recommended by the instrument manufacturer (NeatCell Program) and using a standard aseptic tubing kit. The lymphocyte enrichment step reduces product related impurities such as RBCs, and granulocytes, enriches and concentrates the mononuclear cells, washes and reduces process related residuals such as Ficoll, and formulates the cells in growth media in preparation for cell activation, as well as achieving consistent cell yield and high cell viability. The closed system minimizes environmental contamination.

The process may be carried out in an ISO 7 area at ambient temperature and all connections may be conducted either using a sterile tubing welder, or carried out in an ISO 5 laminar flow hood.

Lymphocyte Activation. The Lymphocyte Activation step may be carried out either with freshly processed cells from the Lymphocyte Enrichment, or previously cryopreserved cells. In the event that cryopreserved cells are used, the cells may be thawed using developed protocols prior to use.

The Lymphocyte Activation step selectively activates lymphocytes to become receptive to retroviral vector transduction, reduces the viable population of all other cell types, achieves consistent cell yield and high lymphocyte viability, and maintains a closed system to minimize the risk of contamination. Lymphocyte Activation can be achieved with lymphocyte stimulating agents such as anti-CD3 antibodies and IL-2.

Wash 1. Following the Lymphocyte Activation step, the cells were washed using cell processing equipment, such as the Sepax® 2 or equivalent, with fresh culture media in a standard aseptic kit using developed protocols by the manufacturer. The cells were optionally concentrated to a final volume of approximately 100 mL in preparation for retroviral vector transduction. The Wash 1 step reduces process related residuals such as anti-CD3 antibody, spent growth media, and cellular debris; achieves consistent cell yield and high T cell viability, maintains a closed system to minimize the risk of contamination; and concentrates and delivers a sufficient number of viable T cells in a small volume appropriate for initiation of transduction.

Transduction. Activated cells from the Wash 1 step in of fresh cell growth media were transferred to a cell culture bag (Origen Biomedical PL240 or comparable) which has been previously prepared by first coating the bags with a recombinant fibronectin or fragments thereof such as RetroNectin® (Takara Bio, Japan), and subsequently incubated with retroviral vector according to defined procedures prior to introduction of the activated cells. RetroNectin® coating (10 μg/mL) was carried out at a temperature of 2-8° C. for 20±4 hr, washed with dilute buffer, and subsequently incubated with thawed retroviral vector for approximately 180-210 min at 37±1° C. and 5±0.5% CO₂. After the addition of cells to the bag, the transduction was carried out for ±4 hr at 37±1° C. and 5±0.5% CO₂. The retroviral transduction step cultures the activated T cells in the presence of the retroviral vector under controlled conditions in order to allow for efficient transduction to take place, achieves consistent cell yield and high cell viability, and maintains a closed system in order to minimize the risk of contamination.

Wash 2. Following the retroviral transduction step, the cells were washed with fresh growth media using cell processing equipment, such as the Sepax® 2 or equivalent, in a standard aseptic kit using protocols developed by the manufacturer, and the cells were concentrated to a final volume of approximately 100 mL in preparation for the expansion step. The Wash 2 step is designed to reduce process related residuals such as retroviral vector particles, vector production process residuals, spent growth media, and cellular debris achieve consistent cell yield and high cell viability; maintain a closed system to minimize the risk of contamination; and exchange spent growth media for fresh media with a target number of cells in a specified volume appropriate for initiation of expansion step.

Lymphocyte Expansion. Cells from the Wash 2 step were aseptically transferred to a culture bag (Origen Biomedical PL325 or equivalent) and diluted with fresh cell growth media and cultured for approximately 72 hr at 37±1° C. and 5±0.5% CO₂. The cell density was measured daily starting on Day 5. Because doubling times of the T cells may vary slightly from subject to subject, additional growth time beyond 72 hr (i.e., 3-6 days) may be necessary in the event that the total cell number is insufficient to deliver a target dose of CAR-positive T cells/kg of subject weight. The lymphocyte expansion step is designed to culture the cells under controlled conditions in order to produce a sufficient number of transduced cells for delivering an efficacious dose, maintain a closed system to minimize risk of contamination, and achieve consistent cell yield and high cell viability. One such efficacious dose or target dose includes 2×10⁶ FMC63-28Z CAR positive or FMC63-CD828BBZ CAR positive T cells/kg (±20%) of subject weight that were produced via transduction with either the MSGV-FMC63-28Z retroviral vector or the MSGV-FMC63-CD828BBZ retroviral vector, respectively, both of which are described in detail in Kochenderfer et al., J Immunother. 2009 September; 32(7): 689-702.

Wash 3 and Concentrate. Following the lymphocyte expansion step, the cells were washed with 0.9% saline using a cell processing instrument, such as the Sepax® 2 or equivalent, in a standard aseptic kit using developed protocols by the manufacturer, and the cells were concentrated to a final volume of approximately 35 mL in preparation for the formulation and cryopreservation. The wash 3 step is designed to reduce process related residuals such as retroviral production process residuals, spent growth media, and cellular debris; achieve consistent cell yield and high cell viability; and maintain a closed system to minimize risk of contamination.

Once the cells have been concentrated and washed into 0.9% saline, an appropriate cell dose may be formulated for preparation of the final cryopreserved product.

The embodiments described herein provide for efficient production of an engineered lymphocyte therapy within 7 days.

### Example 2: Five Day Lymphocyte Manufacturing Process

This example developed an expedited process based on the 7-day process as described in Example 1.

As used throughout, the terms “5-day process” and “5-day lymphocyte manufacturing process” are used interchangeably and refer to a CAR cell manufacturing process which takes about 5 days following initial enrichment and activation steps. The 5-day process is 6 days in length from the initial enrichment and activation steps to a harvesting step, and can be between 6 to 9 days in total when including the enrichment and activation steps.

During the 7-day process, lymphocytes are enriched and activated on day 0; transduction bag is coated with recombinant fibronectin on day 1; viral transduction is conducted on day 2; transduced lymphocytes are washed and then expanded on days 3 and 4; expansion continues with media changed each day on day 5 and 6; and the final cell products are harvested on day 7. From about 1.2×10⁹ lymphocytes acquired from apheresis, about 2.4×10⁸ lymphocytes are incubated with the viral vectors for transduction.

In the newly developed 5-day process, no change was made to the process on day 0. On days 1 and 2, however, a larger bag was used. Instead of the Origen Biomedical PL240 bag being used for transduction, the Origen Biomedical PL325 bag was used, or more preferably, a PL750 bag. The larger bag allowed a larger volume of the vector (200 mL instead of 100 mL) and more lymphocytes (between 3.2×10⁸ and 6×10⁸ instead of 2.4×10⁸) used in the transduction step.

Interestingly, the increased volume of transduction, along with more vectors and starting lymphocytes, did not result in unacceptable reduction of transduction efficiency (54% to 35.15%) or cell viability (92% to 92.4%) (see Table 1). Accordingly, the cell expansion step, which required 4 days in the 7-day process, was reduced to 2 days, enabling harvesting of the final cell products on day 5.

TABLE 1
Evaluation of 5-Day Process vs. 7-Day Process

| Attribute | 7-day average | 5-day average |
|---|---|---|
| Viability (%) | 92 | 92.4 |
| VCN (copies/cell) | 0.4 | 1.21 |
| CD3+ (%) | 91 | 95.83 |
| Transduction rate (%) | 54 | 35.15 |
| IFN-gamma (pg/mL) | 5942 | 2565 |

The modest reduction of transduction rate, however, did not correlate to clinical efficacy or patient safety. Also importantly, it was found that the cell products from the 5-day process included increased percentages of juvenile cells among both CD4+ and CD8+ T cell populations (Table 2) which are believed to be associated with improved therapeutic efficacy. These variations, it is noted, are within historical ranges of donor runs collected from the 7-day process. Note that in Table 2 below, historical averages from 7-day process runs are reflected in the column labeled “Historical averages”, and data from the 5-day and 7-day runs conducted in the current study are depicted in the columns labeled “5-day” and “7-day,” respectively.

TABLE 2
5-Day Skewed Towards Juvenile Cells within Historical Ranges

| | Historical | | |
|---|---|---|---|
| % of cell types | averages | 5-Day | 7-Day |
| CD4 + CM | 54.2 | 50.28 | 45.5 |
| CD4 + Naïve | 18.2 | 40.65 | 24.6 |
| CD4 + Teff/TEMRA | 1.33 | 0.525 | 1.65 |
| CD4 + EM | 26.3 | 8.55 | 28.3 |
| CD8 + CM | 63.4 | 79.8 | 59.4 |
| CD8 + Naïve | 2.04 | 3.93 | 1.6 |
| CD8 + Teff/TEMRA | 0.50 | 0.4 | 0.33 |
| CD8 + EM | 34.0 | 15.85 | 38.7 |

CM: central memory T cells; Teff: T effector cells; EM: effector memory T cells.

This example, therefore, demonstrates that the shortened 5-day process met specification requirements for transduction efficiency, efficacy and safety. Meanwhile, the 5-day products exhibited a more juvenile phenotype within the historical ranges.

The in vivo efficacy of the cell products from the 5-day process was tested in a mouse model implanted with 5×10⁵ Nalm6 cells (a human acute lymphoblastic leukemia (ALL) line) in a 100 μL solution, iv. The CAR used, injected on day 6, targeted CD19, at doses of 5×10⁶ cells, 1×10⁶ cells, or 0.2×10⁶ cells/kg body weight (Table 3).

TABLE 3
Experiment Groups

| Group | N | Primary cells | CAR+ dose/kg |
|---|---|---|---|
| 1 | 5 | Vehicle | N/A |
| 2 | 5 | NTD D 5 | 5 × 10⁶ |
| 3 | 5 | CD19 CAR D 5 | 5 × 10⁶ |
| 4 | 5 | CD19 CAR D 5 | 1 × 10⁶ |
| 5 | 5 | CD19 CAR D 5 | 0.2 × 10⁶ |
| 6 | 5 | CD19 CAR D 5 | 1 × 10⁶ D 7 |
| | | | CAR match |
| 7 | 5 | NTD D 7 | 5 × 10⁶ |
| 8 | 5 | CD19 CAR D 7 | 5 × 10⁶ |
| 9 | 5 | CD19 CAR D 7 | 1 × 10⁶ |
| 10 | 5 | CD19 CAR D 7 | 0.2 × 10⁶ |

Body weight changes (Table 4), which correlated to increased tumor burden, indicate that all these treatments were safe to the animals.

TABLE 4
Changes of Body Weights at Days Post Tumor Implantation

| Day | G1 | G2 | G3 | G4 | G5 | G6 | G7 | G8 | G9 | G10 |
|---|---|---|---|---|---|---|---|---|---|---|
| 5 | 0.3 | −0.68 | 0.98 | 0.28 | 0.08 | −1 | −1.36 | 0.8 | −2.14 | 0.28 |
| 8 | 3.98 | 3.26 | 5.68 | 4.5 | 2.46 | 4.28 | 2.68 | 4.14 | 2.38 | 4.22 |
| 13 | 5.22 | 2.3 | 6.22 | 4.74 | 3.96 | 2.1 | 1.98 | 4.74 | 1.38 | 3.22 |
| 16 | 6.58 | 4.62 | 9.44 | 8.08 | 5.74 | 5.68 | 5.5 | 7.28 | 3.24 | 6.84 |
| 20 | 3.28 | 0 | 7.08 | 5.8 | 5 | 3.76 | 1.96 | 3.62 | 2.28 | 5.68 |
| 23 | −0.38 | −6.58 | 10.3 | 7.82 | 8.66 | 7.2 | −7 | 7.7 | 6.04 | 9 |
| 26 | −5.9 | −12.4 | 11.1 | 8.54 | 9.46 | 9.06 | −15.8 | 9.1 | 7.22 | 11.48 |
| 29 | | −21.4 | 10.2 | 9.02 | 9.6 | 8.88 | | 8.08 | 5.44 | 8.66 |
| 33 | | | 11.6 | 9.36 | 9.22 | 11.06 | | 9.18 | 7.56 | 9.78 |
| 40 | | | 8.76 | 13.28 | 12.74 | 14.1 | | 13.38 | 10.78 | 12.62 |
| 43 | | | 5.28 | 14.12 | 13.52 | 14.34 | | 13.16 | 12.1 | 8.1 |
| 47 | | | 8.43 | 12.94 | 13.18 | 13.36 | | 11.38 | 11.78 | 1.4 |
| 50 | | | 3.58 | 12.2 | 11.22 | 14.96 | | 10.32 | 12.42 | 13.95 |
| 54 | | | 1.53 | 7.5 | 9.05 | 12.26 | | 8.675 | 8.48 | 13.85 |
| 57 | | | −0.17 | 8.6 | 8.475 | 13.58 | | 9.275 | 6.68 | 15.7 |
| 61 | | | −2 | 11.18 | 4.325 | 14.36 | | 10.65 | 13.53 | 17.3 |

The tumor inhibition results are shown in Table 5 (measured as photons/s).

TABLE 5
Tumor Mass at Days Post Tumor Implantation

| Day | G1 | G2 | G3 | G4 | G5 | G6 | G7 | G8 | G9 | G10 |
|---|---|---|---|---|---|---|---|---|---|---|
| 5 | 3.7e06 | 6.1e06 | 5.1e06 | 5.3e06 | 2.8e06 | 2.0e06 | 7.5e06 | 7.0e06 | 6.7e06 | 4.7e06 |
| 8 | 6.1e07 | 1.3e08 | 1.5e06 | 1.8e07 | 3.2e07 | 1.7e07 | 9.8e07 | 1.3e06 | 1.5e07 | 6.4e07 |
| 13 | 7.7e09 | 9.5e09 | 8.1e05 | 7.2e05 | 6.9e06 | 7.5e05 | 7.0e09 | 6.7e05 | 8.1e05 | 1.5e08 |
| 16 | 1.3e10 | 1.6e10 | 7.1e05 | 7.1e05 | 7.3e05 | 8.0e05 | 1.8e10 | 7.6e05 | 8.9e05 | 2.7e07 |
| 20 | 2.2e10 | 3.2e10 | 6.3e05 | 6.5e05 | 7.5e05 | 7.2e05 | 3.0e10 | 6.9e05 | 8.2e05 | 1.1e06 |
| 23 | 5.1e10 | 6.4e10 | 7.7e05 | 7.6e05 | 8.7e05 | 8.0e05 | 3.8e10 | 7.7e05 | 1.6e06 | 2.0e06 |
| 26 | 4.2e10 | 1.1e11 | 7.9e05 | 7.9e05 | 1.4e06 | 9.9e05 | 1.4e11 | 7.6e05 | 4.3e06 | 8.7e06 |
| 29 | | 1.9e11 | 6.3e05 | 6.2e05 | 1.6e06 | 1.0e06 | | 7.4e05 | 3.3e06 | 8.6e06 |
| 33 | | | 6.3e05 | 7.1e05 | 9.2e06 | 3.3e06 | | 1.4e06 | 2.9e07 | 2.8e08 |
| 35 | | | 7.1e05 | 9.2e05 | 1.3e07 | 1.4e07 | | 6.3e06 | 1.2e08 | 8.2e08 |
| 40 | | | 7.2e05 | 2.6e06 | 2.0e08 | 6.0e07 | | 3.0e07 | 1.1e09 | 7.2e09 |
| 43 | | | 7.6e05 | 6.0e06 | 1.2e09 | 4.4e07 | | 1.8e08 | 3.4e09 | 1.0e10 |
| 47 | | | 9.1e05 | 1.1e07 | 2.5e09 | 2.8e08 | | 5.2e06 | 1.6e09 | 6.2e09 |
| 50 | | | 8.1e05 | 8.6e06 | 7.1e09 | 1.4e09 | | 2.1e06 | 8.0e09 | 3.2e09 |
| 54 | | | 9.4e05 | 2.6e06 | 1.3e10 | 4.6e09 | | 1.4e06 | 9.9e09 | 4.4e09 |
| 57 | | | 9.0e05 | 1.9e06 | 8.4e09 | 7.1e09 | | 1.2e06 | 9.0e09 | 4.9e09 |
| 61 | | | 9.3e05 | 1.5e06 | 5.3e09 | 1.5e09 | | 9.7e05 | 2.7e09 | 9.3e09 |

When dosed at 5×10⁶ cells/kg, the cell products from the 5-day process showed complete tumor growth inhibition. At all doses, the cells products from the 5-day process outperformed those from the 7-day process. Cells from the 5-day process were also more efficacious than those of the 7-day process, even when the percentage of CAR+ cells was matched (i.e., fewer CAR-T cells from the 5-day process).

### Example 3: Three-Day Lymphocyte Manufacturing Process

This example developed a further expedited process based on the 7-day process as described in Example 1 and the 5-day process as described in Example 2.

As used throughout, the terms “3-day process” and “3-day lymphocyte manufacturing process” are used interchangeably and refer to a CAR cell manufacturing process which takes about 3 days from initial enrichment and activation steps. The 3-day process is about 4 days in length from the initial enrichment and activation step to a harvesting step. The 3-day process does not include a cell expansion step comprising one or more days following a transduction step and preceding a harvesting step.

In this new process, the day 0-1 procedures are similar to the 5-day process, including the fibronectin-coating of a larger bag (e.g., Origen Biomedical PL325 or preferably PL750) for the subsequent transduction on day 2. On day 2, however, only about 4.8×10⁸ (instead of 6×10⁸) lymphocytes were used in the transduction step, with the same amount of viral vectors (200 mL). Another important difference is that, unlike in the 5-day and 7-day processes, no specific step of T cell expansion was conducted. Instead, the transduced lymphocytes were harvested on day 3, allowing the entire process to be completed within 3 days from the initial enrichment and activation steps.

Given the lack of a specific T cell expansion step in this newly developed 3-day process, the cell products harvested included a slightly smaller percentage of T cells (CD3+). Importantly, however, the 3-day products included an even greater percentage of juvenile (naïve) T cells (Table 6). Note that in Table 6 below, historical averages from 7-day process runs are reflected in the column labeled “Historical averages”, and data from the 3-day and 7-day runs conducted in the current study are depicted in the columns labeled “3-day” and “7-day,” respectively.

TABLE 6
Phenotypes of the Cell Products

| | Historical | | |
|---|---|---|---|
| | averages | 3-Day | 7-Day |
| CD4 + CM | 54.2 | 39.65 | 44.55 |
| CD4 + Naïve | 18.2 | 55.75 | 26.75 |
| CD4 + Teff/TEMRA | 1.33 | 0.25 | 1.65 |
| CD4 + EM | 26.3 | 4.35 | 27 |
| CD8 + CM | 63.4 | 46.95 | 63.05 |
| CD8 + Naïve | 2.04 | 37.35 | 2.6 |
| CD8 + Teff/TEMRA | 0.50 | 5.9 | 0.5 |
| CD8 + EM | 34.0 | 9.85 | 33.85 |

CM: central memory T cells; Teff: T effector cells; EM: effector memory T cells.

Even though not tested with lymphocytes from the same donors, comparison between Tables 2 and 6 indicate that the naïve T cell percentages from the 3-day process were also significantly higher than from the 5-day process. Within CD4+ T cells, the 3-day process generated about 55.75% naïve T cells while the 5-day process generated about 40.65%; within CD8+ T cells, the 3-day process generated about 37.35% naïve T cells while the 5-day process generated about 3.93%.

Put in other terms, the 3-day process generates roughly a 1.4-fold increase in the percentage of CD4+ naïve T cells versus the percentage of such cells observed from the 5-day process, and roughly an 9.5-fold increase in the percentage of CD8+ naïve T cells versus the percentage of such cells observed from the 5-day process. Also, the 3-day process generates roughly a 3.0-fold increase in the percentage of CD4+ naïve T cells versus the historical average of such cells observed from the 7-day process, and roughly an 18.0-fold increase in the percentage of CD8+ naïve T cells versus the historical average of such cells observed from the 7-day process.

Inversely, within CD4+ T cells, the 3-day process generated only about 4.35% effector memory T cells while the 5-day process generated about 8.55%; within CD8+ T cells, the 3-day process only generated about 9.85% effector memory T cells while the 5-day process generated about 15.85%.

Put in other terms, the 3-day process generates roughly a 2.0-fold decrease in the percentage of CD4+ effector memory T cells versus the percentage of such cells observed from the 5-day process, and roughly an 1.6-fold decrease in the percentage of CD8+ effector memory T cells versus the percentage of such cells observed from the 5-day process. Also, the 3-day process generates roughly a 6.0-fold decrease in the percentage of CD4+ effector memory T cells versus the historical average of such cells observed from the 7-day process, and roughly a 3.5-fold decrease in the percentage of CD8+ effector memory T cells versus the historical average of such cells observed from the 7-day process.

Also, as can be appreciated from Table 6, the percentage of CD4+ CCR7+ cells (i.e. CM and Naive cells) in the final harvested population from the 3-day process is at least 95% of the harvested CD4+ T-cell population. Put another way, the maximum percentage of Teff/TEMRA and EM CD4+ cells in the final harvested population from the 3-day process is about 5%. Thus, there are at least 19 times as many CD4+ CCR7+ cells in the harvested population as Teff/TEMRA and EM CD4+ cells. By comparison, the percentage of CD4+ CCR7+ cells (i.e. CM and Naive cells) in the final harvested population from the 7-day process is about 71% of the harvested CD4+ T-cell population. Put another way, the maximum percentage of Teff/TEMRA and EM CD4+ cells in the final harvested population from the 7-day process is about 29%. Thus, the 3-day process generates a population of CD4+ T-cells with about a 1.3-fold increase in the percentage of CD4+ CCR7+ cells as the 7-day process, and with about a 5-fold decrease in the percentage of Teff/TEMRA and EM CD4+ cells.

As also seen in Table 6, the percentage of CD8+ CCR7+ cells (i.e. CM and Naive cells) in the final harvested population from the 3-day process is at least 84% of the harvested CD8+ T-cell population. Put another way, the maximum percentage of Teff/TEMRA and EM CD4+ cells in the final harvested population from the 3-day process is about 16%. Thus, there are at least about 5 times as many CD8+ CCR7+ cells in the harvested population as Teff/TEMRA and EM CD4+ cells. By comparison, the percentage of CD8+ CCR7+ cells (i.e. CM and Naive cells) in the final harvested population from the 7-day process is about 66% of the harvested CD8+ T-cell population. Put another way, the maximum percentage of Teff/TEMRA and EM CD8+ cells in the final harvested population from the 7-day process is about 34%. Thus, the 3-day process generates a population of CD8+ T-cells with about a 1.3-fold increase in the percentage of CD8+ CCR7+ cells as the 7-day process, and with about a 2-fold decrease in the percentage of Teff/TEMRA and EM CD8+ cells.

Cell viability measurements showed that, throughout the 3-day process, cell viability stayed high (>90%), while the wash step on day 2 caused the most reduction on cell viability. By contrast, the 5-day and 7-day processes included additional washing steps, each of which contributed to additional drop in cell viability (Table 7).

TABLE 7
Cell Viability Changes During Process

| Day/Viability % | 3 Day | 7 Day (Ctrl) |
|---|---|---|
| D 0 Aph | 99.3 | 99.3 |
| D 0 - post neat | 99.1 | 99.1 |
| D 0 - post seed | 99.2 | 99 |
| D 2 - post wash | 92.9 | 92.2 |
| D 2 - post seed | 91 | 92.7 |
| D 3 - pre wash | 92.6 | 94.4 |
| D 3 - post wash | 81.5 | 89.2 |
| D 3 - post seed | | 87.6 |
| D 5 | | 97.6 |
| D 6 | | 97.8 |
| D 7 - pre wash | | 97.1 |
| D 7 - post wash | | 92.6 |

### Example 4. In Vivo Efficacy of Cells Prepared by the Three-Day Process

This example compared the in vivo efficacy of cells prepared by the 3-day process as described in Example 3, to those from the 7-day process, with the same animal model and procedure as described in Example 2.

The experiment groups are shown in Table 8, and the body weight changes are shown in Table 9.

TABLE 8
Experiment Groups

| Group | N | Primary cells | CAR+ dose/kg |
|---|---|---|---|
| 1 | 5 | Vehicle | Vehicle |
| 2 | 5 | NTD D 3 | 3 × 10⁶ |
| 3 | 5 | NTD D 3 | 0.5 × 10⁶ |
| 4 | 5 | NTD D 7 | 3 × 10⁶ |
| 5 | 5 | NTD D 7 | 0.5 × 10⁶ |
| 6 | 5 | CD19 CAR D 3 | 3 × 10⁶ |
| 7 | 5 | CD19 CAR D 3 | 1 × 10⁶ |
| 8 | 5 | CD19 CAR D 3 | 0.5 × 10⁶ |
| 9 | 5 | CD19 CAR D 7 | 3 × 10⁶ |
| 10 | 5 | CD19 CAR D 7 | 1 × 10⁶ |
| 11 | 5 | CD19 CAR D 7 | 0.5 × 10⁶ |

TABLE 9
Changes of Body Weights at Days Post Tumor Implantation

| Day | G1 | G2 | G3 | G4 | G5 | G6 | G7 | G8 | G9 | G10 | G11 |
|---|---|---|---|---|---|---|---|---|---|---|---|
| 5 | 0.3 | 0.64 | 1.54 | 0.48 | −0.6 | −0.1 | 0.02 | −0.16 | 0.76 | 0.46 | 0.3 |
| 8 | 6.46 | 5.78 | 10.98 | 9.62 | 7.72 | 7.06 | 5.74 | 4.8 | 7.84 | 8.2 | 5.6 |
| 12 | 11.58 | 11.62 | 13.62 | 13.42 | 11.54 | 11.76 | 10.08 | 8.32 | 13.88 | 10.82 | 9.24 |
| 15 | 13.26 | 12.78 | 14.88 | 15.1 | 13.52 | 12.42 | 11.94 | 11.38 | 14.6 | 13.18 | 10.84 |
| 20 | −4.78 | −4.52 | −1.43 | −0.54 | −9.95 | 7.42 | 13.18 | 10.7 | 14.34 | 10.6 | 13.1 |
| 23 | | | | | | 3.48 | 12.52 | 10.26 | 12.8 | 10.2 | 12.38 |
| 26 | | | | | | 3.5 | 13.94 | | 13.4 | 11.62 | 11.18 |
| 34 | | | | | | −8.13 | 12.24 | 7.56 | 10.5 | 6.36 | −8.95 |
| 40 | | | | | | −10.63 | 7.16 | 10.3 | 11.7 | −1.2 | |
| 47 | | | | | | | 1.72 | 15.98 | 5.28 | | |
| 61 | | | | | | | | 7.7 | 10.68 | | |
| 68 | | | | | | | 19.25 | 10.07 | 11.1 | | |

The tumor inhibition results are shown in Table 10 (measured as photons/s).

TABLE 10
Tumor Mass at Days Post Tumor Implantation

| Day | G1 | G2 | G3 | G4 | G5 | G6 | G7 | G8 | G9 | G10 | G11 |
|---|---|---|---|---|---|---|---|---|---|---|---|
| 5 | 2.5e06 | 2.0e06 | 2.5e06 | 1.8e06 | 2.0e06 | 1.5e06 | 2.8e06 | 1.8e06 | 1.4e06 | 1.8e06 | 1.4e06 |
| 8 | 2.3e07 | 2.2e07 | 2.3e07 | 9.5e06 | 1.7e07 | 4.4e06 | 1.4e07 | 1.4e07 | 7.5e05 | 2.0e06 | 3.9e06 |
| 12 | 4.8e08 | 3.0e08 | 4.4e08 | 4.1e08 | 6.7e08 | 6.9e05 | 1.7e06 | 3.2e06 | 6.8e05 | 8.0e05 | 2.7e06 |
| 15 | 4.1e09 | 3.6e09 | 4.1e09 | 1.7e09 | 6.6e09 | 7.2e05 | 6.8e05 | 1.1e06 | 8.2e05 | 1.3e06 | 8.8e06 |
| 20 | 1.6e10 | 1.7e10 | 2.7e10 | 1.3e10 | 1.7e10 | 7.4e05 | 6.8e05 | 7.7e05 | 8.0e05 | 8.8e06 | 8.2e07 |
| 23 | | | | 3.1e10 | 3.1e10 | 7.7e05 | 6.2e05 | 7.2e05 | 6.7e05 | 3.1e07 | 1.7e08 |
| 26 | | | | | | 9.8e05 | 6.6e05 | 6.8e05 | 2.3e06 | 4.2e08 | 2.4e09 |
| 30 | | | | | | 1.0e06 | 7.5e05 | 8.5e05 | 1.1e08 | 3.9e09 | 1.4e10 |
| 34 | | | | | | 7.2e05 | 6.3e05 | 6.2e05 | 8.5e08 | 7.2e09 | 2.3e10 |
| 37 | | | | | | 7.8e05 | 7.8e05 | 7.9e05 | 1.9e09 | 1.4e10 | 2.3e10 |
| 40 | | | | | | 8.9e05 | 9.4e05 | 9.4e05 | 4.2e09 | | |
| 47 | | | | | | | 6.3e05 | 8.7e05 | 1.1e10 | | |
| 61 | | | | | | | 8.6e05 | 1.0e06 | 6.8e09 | | |
| 68 | | | | | | | 6.7e05 | 8.6e05 | 5.2e09 | | |

At all three doses (0.5×, 1×, and 3×10⁶ cells/kg), cell products from the 3-day process were able to completely inhibit tumor growth throughout the observation period, without marked differences. By contrast, the cells from the 7-day process were only able to inhibit tumor growth through 15 days (0.5 and 1×10⁶ cells/kg), and 23 days (3×10⁶ cells/kg).

This example, therefore, demonstrates that the 3-day process is able to prepare cell products with greatly improved antitumor efficacy as compared to the 7-day process. Moreover, given the greatly improved efficacy, a much lower dose is required to achieved a much more complete antitumor response.

In a further experiment, an even lower dose of the cells from the 3-day process (0.2×10⁶ cells/kg; three different donors) were compared to a high dose (1×10⁶ cells/kg) from the 7-day process. The results (Table 11) shows that, even at a 5-fold difference, the 3-day process's results were far superior to the 7-day one.

TABLE 11
Tumor Mass at Days Post Tumor Implantation

| | | 7-Day at | 3-Day at | 3-Day at | 3-Day at |
|---|---|---|---|---|---|
| Day | Vehicle | 1 × 10⁶ | 0.2 × 10⁶ | 0.2 × 10⁶ | 0.2 × 10⁶ |
| 6 | 5.63e07 | 7.7e07 | 9.2e07 | 1.0e08 | 5.1e07 |
| 12 | 1.45e10 | 3.7e07 | 6.5e09 | 1.5e10 | 1.3e10 |
| 15 | 3.09e10 | 2.5e06 | 7.6e08 | 1.9e09 | 8.2e09 |
| 19 | 7.61e10 | 1.2e06 | 1.3e06 | 1.6e06 | 1.4e06 |
| 22 | 1.02e11 | 1.2e06 | 1.2e06 | 1.4e06 | 1.5e06 |
| 26 | | 2.6e06 | 1.2e06 | 1.3e06 | 1.5e06 |
| 29 | | 4.9e06 | 1.0e06 | 1.4e06 | 1.4e06 |
| 33 | | 6.0e06 | 1.1e06 | 1.3e06 | 1.5e06 |
| 36 | | 9.9e06 | 1.0e06 | 1.3e06 | 1.3e06 |
| 40 | | 4.1e07 | 9.2e05 | 9.2e05 | 1.3e06 |
| 43 | | 1.9e08 | 1.3e06 | 1.4e06 | 1.8e06 |
| 47 | | 1.8e09 | 1.0e06 | 1.2e06 | 2.3e06 |
| 54 | | 3.5e10 | 9.5e05 | 1.2e06 | 3.6e06 |
| 61 | | 7.4e10 | 9.4e05 | 1.6e06 | 1.4e07 |
| 68 | | 4.4e09 | 1.1e06 | 1.4e06 | 3.2e07 |
| 75 | | 1.1e10 | 1.1e06 | 1.2e06 | 6.3e07 |
| 82 | | 2.6e10 | 1.1e06 | 1.0e06 | 2.1e08 |
| 89 | | 9.7e09 | 1.0e06 | 1.0e06 | 6.3e09 |

### Example 5: Impact of Earlier Harvesting on CAR

This example discovered that the improved performance of the abbreviated (earlier harvesting) process was more pronounced when the CAR construct included a CD28 co-stimulatory domain, as compared to a 4-1BB co-stimulatory domain.

Both types of CAR included a CD19-binding antigen binding fragment, while CAR #1 included a CD28 co-stimulatory domain, and CAR #2 included a 4-1BB co-stimulatory domain, both delivered by a lentiviral vector. Cells were harvested at day 4, day 7 and day 14, and doses for the in vivo animal study included 0.2×10⁶ cells/kg and 1×10⁶ cells/kg (Table 12). The results are shown in Table 13.

TABLE 12
Experiment Groups

| Group | N | Primary cells | CAR+ dose/kg |
|---|---|---|---|
| G1 | 5 | Vehicle | Vehicle |
| G2 | 5 | NTD_D 14 1e6 | 1 × 10⁶ |
| G3 | 5 | CD19-CD28_D 4 | 0.2 × 10⁶ |
| G4 | 5 | CD19-CD28_D 7 | 1 × 10⁶ |
| G5 | 5 | CD19-CD28_D 7 | 0.2 × 10⁶ |
| G7 | 5 | CD19-CD28_D 14 | 0.2 × 10⁶ |
| G8 | 5 | CD19-41BB_D 4 | 0.2 × 10⁶ |
| G9 | 5 | CD19-41BB_D 7 | 1 × 10⁶ |
| G10 | 5 | CD19-41BB_D 7 | 0.2 × 10⁶ |
| G12 | 5 | CD19-41BB_D 14 | 0.2 × 10⁶ |

TABLE 13
Tumor Mass at Days Post Tumor Implantation

| Day | G1 | G2 | G3 | G4 | G5 | G7 | G8 | G9 | G10 | G12 |
|---|---|---|---|---|---|---|---|---|---|---|
| 5 | 3.1e06 | 2.5e06 | 1.3e06 | 1.2e06 | 1.7e06 | 3.4e06 | 2.3e06 | 8.3e05 | 1.1e06 | 1.1e06 |
| 8 | 3.5e07 | 2.6e07 | 6.0e06 | 1.0e06 | 7.0e06 | 2.4e07 | 2.5e07 | 1.3e06 | 3.9e06 | 3.6e06 |
| 11 | 7.8e08 | 5.2e08 | 2.1e07 | 7.4e05 | 1.1e07 | 7.8e07 | 1.5e08 | 7.5e05 | 9.1e06 | 3.8e06 |
| 14 | 5.7e09 | 6.1e09 | 1.5e06 | 1.2e06 | 5.8e06 | 5.7e07 | 4.6e07 | 8.7e05 | 7.3e06 | 3.6e06 |
| 18 | | | 7.9e05 | 1.0e06 | 2.0e06 | 4.0e07 | 3.3e06 | 9.7e05 | 1.8e07 | 1.9e07 |
| 21 | | | 8.0e05 | 1.8e06 | 6.1e06 | 4.3e07 | 1.1e06 | 1.3e06 | 2.0e07 | 1.1e08 |
| 25 | | | 9.9e05 | 7.7e06 | 7.9e07 | 2.0e08 | 1.4e06 | 1.7e06 | 5.7e07 | 5.3e08 |
| 28 | | | 1.2e06 | 4.7e07 | 6.4e08 | 1.6e09 | 3.6e06 | 6.1e06 | 4.7e08 | 3.1e09 |
| 32 | | | 9.5e05 | 3.2e08 | 5.7e09 | | 1.8e07 | 4.6e07 | 5.5e09 | |
| 35 | | | 1.2e06 | 2.5e09 | 9.3e09 | | 7.9e07 | 3.7e08 | 1.6e10 | |
| 39 | | | 3.3e06 | | | | 1.2e09 | 1.1e09 | | |
| 42 | | | 1.3e07 | | | | 8.4e08 | 9.5e08 | | |
| 46 | | | 9.9e07 | | | | 9.8e06 | 3.3e08 | | |
| 49 | | | 5.3e08 | | | | 2.9e07 | 6.6e08 | | |
| 53 | | | 2.9e08 | | | | 3.9e08 | 1.1e09 | | |
| 56 | | | 1.1e07 | | | | 2.7e09 | | | |
| 60 | | | 8.2e06 | | | | 1.0e10 | | | |
| 62 | | | 1.3e07 | | | | | | | |
| 67 | | | 1.5e07 | | | | | | | |
| 70 | | | 7.1e06 | | | | | | | |
| 74 | | | 2.3e06 | | | | | | | |
| 77 | | | 2.6e06 | | | | | | | |
| 81 | | | 2.8e06 | | | | | | | |
| 84 | | | 1.7e07 | | | | | | | |
| 88 | | | 8.4e07 | | | | | | | |

As shown in Table 13, with late harvesting (day 7 or 14), the difference of performance between the two constructs was limited. With the early harvesting which benefited both constructs, however, CAR #1 (G3) exhibited significantly higher in vivo efficacy than CAR #2 (G8).

Additionally, a CAR that targets CD19 and includes a CD28 costimulatory domain was tested in vivo with the Nalm6 mouse model of leukemia. The results showed that early harvesting (day 4) led to better in vivo anti-tumor efficacy than regular harvesting (day 7), which in turn was more efficacious than late harvesting (day 14). In fact, a low dose (2×10⁵ cells) of T cells harvested on day 4 was 5 times more efficacious than a high dose (10⁶ cells) of T cells harvested on day 7.

### Example 6 In Vivo Efficacy of Cells Prepared by the Three Day Process

This example complements Example 4 and presents additional data comparing the in vivo efficacy of anti-CD19 CART cells prepared by the 3-day process described in Example 3, to those prepared by the 7-day process.

Anti-CD19 CAR T cells prepared by the 3-day process and anti-CD19 CAR T cells prepared by the 7-day process along with their respective non-transduced (NTD) Day 3 and NTD Day 7 cells were manufactured from a healthy human donor. NALM6-luc cells (5×10⁵ cells) were implanted IV via the lateral tail vein into 8-week-old female NSG mice (5 mice per group). On Day 6 after tumor implantation, mice received either controls (NTD Day 3 or NTD Day 7) in a single dose cohort or anti-CD19 CAR T-cell products as multiple dose cohorts. A total of 5 dose cohorts (1×10⁶, 5×10⁵, 3×10⁵, 2×10⁵, and 1×10⁵ CAR⁺ T cells) were tested for cells derived from the 3-day process, and 4 dose cohorts (1×10⁷, 5×10⁶, 3×10⁶, and 1×10⁶ CAR⁺ T cells) were tested for cells derived from the 7-day process. Antitumor activity was assessed by determining the effects of anti-CD19 CAR T-cell products on tumor burden and animal survival.

Antitumor activity was evaluated by assessing the change in tumor burden as measured by bioluminescence imaging (BLI) from Day 5 (1 day before CAR T-cell treatment) to Day 22 (16 days after CAR T-cell treatment, and the last time point in which all groups were intact). Inhibition of tumor growth was observed in all 4 dose cohorts of mice treated with anti-CD19 CAR T cells derived from the 7-day process and all 5 dose cohorts of anti-CD19 CAR T cells derived from the 3-day process (Tables 14, 15, and 16). For anti-CD19 CAR T cells derived from the 3-day process, tumor growth inhibition compared with the no treatment group was 128.1%, 126.7%, 117.7%, 113.4%, and 65.7% in animals treated with 1×10⁶, 5×10⁵, 3×10⁵, 2×10⁵, and 1×10⁵ CAR⁺ T cells, respectively. For the anti-CD19 CAR T cells derived from the 7-day process, tumor growth inhibition ranged between 109.0% to 115.1% for the dose cohorts tested (Table 16). Tumor burden regressed at Day 22 in all groups and at all CAR′ T-cell doses tested except for the 1×10⁵ CAR⁺ T-cell dose cohort of anti-CD19 CAR T cells prepared by the 3-day process, for which delayed tumor control occurred by Day 26. The antitumor efficacy at all doses of anti-CD19 CAR T cells prepared by the 3-day process and anti-CD19 CAR T cells prepared by the 7-day process reached statistical significance on Day 22 when compared with the mice that received no treatment or NTD controls (Tukey's post-test, p<0.05 was considered significant).

TABLE 14
Experiment Groups for Results presented in Tables 15 and 16

| Group | N | Primary cells | CAR+ dose/kg |
|---|---|---|---|
| G1 | 5 | No treatment | N/A |
| G2 | 5 | NTD_D 3 | 1 × 10⁶ |
| G3 | 5 | NTD_D 7 | 1 × 10⁷ |
| G4 | 5 | CD19-CD28_D 3 | 1 × 10⁵ |
| G5 | 5 | CD19-CD28_D 3 | 2 × 10⁵ |
| G6 | 5 | CD19-CD28_D 3 | 3 × 10⁵ |
| G7 | 5 | CD19-CD28_D 3 | 5 × 10⁵ |
| G8 | 5 | CD19-CD28_D 3 | 1 × 10⁶ |
| G9 | 5 | CD19-CD28_D 7 | 1 × 10⁶ |
| G10 | 5 | CD19-CD28_D 7 | 3 × 10⁶ |
| G11 | 5 | CD19-CD28_D 7 | 5 × 10⁶ |
| G12 | 5 | CD19-CD28_D 7 | 1 × 10⁷ |

TABLE 15
Tumor Mass at Days Post Tumor Implantation (presented as the average value observed for each group per timepoint; units = photons/s)

| Day | G1 | G2 | G3 | G4 | G5 | G6 | G7 | G8 | G9 | G10 | G11 | G12 |
|---|---|---|---|---|---|---|---|---|---|---|---|---|
| 5 | 2.10E+06 | 4.76E+06 | 3.22E+06 | 2.24E+06 | 2.23E+06 | 3.52E+06 | 7.51E+06 | 8.42E+06 | 2.28E+06 | 6.95E+06 | 2.70E+06 | 2.64E+06 |
| 8 | 5.87E+06 | 3.64E+07 | 2.32E+07 | 8.95E+06 | 9.52E+06 | 8.38E+06 | 6.03E+07 | 1.07E+08 | 1.21E+07 | 5.68E+06 | 9.15E+05 | 6.19E+05 |
| 13 | 4.04E+09 | 8.69E+09 | 2.10E+09 | 1.81E+08 | 1.18E+07 | 2.03E+06 | 1.66E+06 | 7.77E+05 | 1.17E+06 | 6.17E+05 | 7.19E+05 | 7.00E+05 |
| 16 | 9.43E+09 | 1.49E+10 | 1.11E+10 | 3.06E+07 | 6.67E+05 | 6.06E+05 | 6.58E+05 | 7.01E+05 | 6.93E+05 | 1.30E+06 | 6.26E+05 | 6.70E+05 |
| 19 | 1.38E+10 | 2.64E+10 | 1.70E+10 | 3.83E+07 | 6.12E+05 | 5.69E+05 | 5.07E+05 | 5.24E+05 | 6.79E+05 | 3.11E+06 | 5.96E+05 | 5.50E+05 |
| 22 | 3.12E+10 | 4.96E+10 | 2.53E+10 | 5.80E+07 | 5.70E+05 | 5.11E+05 | 5.44E+05 | 5.70E+05 | 9.69E+05 | 5.05E+06 | 6.55E+05 | 6.06E+05 |
| 26 | | 8.22E+10 | 9.81E+10 | 6.19E+05 | 6.32E+05 | 5.64E+05 | 5.70E+05 | 5.47E+05 | 6.81E+06 | 4.54E+06 | 7.32E+05 | 6.30E+05 |
| 29 | | | | 4.73E+05 | 5.06E+05 | 4.54E+05 | 4.80E+05 | 5.37E+05 | 1.23E+07 | 1.16E+07 | 1.21E+06 | 5.85E+05 |
| 33 | | | | 4.98E+05 | 5.31E+05 | 6.04E+05 | 5.91E+05 | 1.29E+06 | 3.04E+08 | 3.37E+07 | 3.75E+06 | 6.65E+05 |
| 36 | | | | 4.66E+05 | 5.25E+05 | 5.86E+05 | 5.67E+05 | 4.18E+06 | 1.55E+09 | 1.21E+08 | 1.87E+07 | 5.60E+05 |
| 40 | | | | 6.06E+05 | 6.52E+05 | 8.47E+05 | 7.41E+05 | 1.47E+07 | 5.75E+09 | 3.11E+08 | 6.73E+07 | 6.92E+05 |
| 43 | | | | 5.70E+05 | 5.79E+05 | 7.31E+05 | 6.16E+05 | 2.19E+07 | 8.45E+09 | 1.10E+09 | 2.48E+08 | 6.90E+05 |
| 47 | | | | 4.77E+05 | 4.16E+05 | 6.66E+05 | 6.63E+05 | 1.33E+07 | 5.27E+09 | 3.59E+09 | 6.25E+08 | 7.02E+05 |
| 50 | | | | 5.28E+05 | 4.58E+05 | 6.94E+05 | 7.34E+05 | 1.78E+07 | 7.42E+09 | 9.60E+09 | 3.55E+09 | 8.75E+05 |
| 54 | | | | 6.66E+05 | 6.25E+05 | 8.24E+05 | 8.70E+05 | 3.39E+07 | 1.48E+10 | 7.90E+09 | 2.83E+09 | 3.07E+06 |
| 57 | | | | 5.72E+05 | 4.19E+05 | 6.15E+05 | 6.68E+05 | 3.79E+07 | 1.66E+10 | 8.29E+09 | 5.05E+09 | 1.17E+07 |
| 61 | | | | 5.54E+05 | 6.59E+05 | 6.42E+05 | 6.41E+05 | 6.26E+05 | 7.12E+09 | 4.72E+09 | 8.92E+08 | 5.74E+07 |
| 64 | | | | 6.08E+05 | 5.06E+05 | 6.29E+05 | 5.88E+05 | 5.39E+05 | 8.40E+09 | 6.19E+09 | 1.26E+09 | 1.22E+08 |

TABLE 16
Log-transform Analysis of Tumor Burden

| | G1 | G2 | G3 | G8 | G7 | G6 | G5 | G4 | G12 | G11 | G10 | G9 |
|---|---|---|---|---|---|---|---|---|---|---|---|---|
| Day 5 | 6.31 | 6.58 | 6.49 | 6.92 | 6.85 | 6.45 | 6.32 | 6.33 | 6.41 | 6.42 | 6.83 | 6.34 |
| Day 22 | 10.48 | 10.69 | 10.38 | 5.75 | 5.73 | 5.71 | 5.75 | 7.76 | 5.78 | 5.81 | 6.25 | 5.97 |
| Change, | 4.17 | 4.11 | 3.89 | −1.17 | −1.11 | −0.74 | −0.56 | 1.43 | −0.63 | −0.61 | −0.58 | −0.38 |
| Day 5 to | | | | | | | | | | | | |
| Day 22 | | | | | | | | | | | | |
| ΔT/ΔC | 100.0 | 98.4 | 93.3 | −28.1 | −26.7 | −17.7 | −13.4 | 34.3 | −15.1 | −14.6 | −13.9 | −9.0 |
| (%) | | | | | | | | | | | | |
| Tumor | 0.0 | 1.6 | 6.7 | 128.1 | 126.7 | 117.7 | 113.4 | 65.7 | 115.1 | 114.6 | 113.9 | 109.0 |
| Growth | | | | | | | | | | | | |
| Inhibition, | | | | | | | | | | | | |
| Day 22 | | | | | | | | | | | | |
| (%) | | | | | | | | | | | | |

In Table 16, tumor burden (measured by BLI) was login normalized, with means for each cohort at Day 5 and Day 20 of the study represented in the table. The change in tumor BLI from Day 5 to Day 20 was calculated, and the percent change in tumor burden relative to the no treatment group is calculated as ΔT/ΔC. Tumor growth inhibition was defined as (1-[ΔT/ΔC]×100) and represented the percentage tumor volume change in treatment arms relative to the control (ie, mice that received no treatment). A Tumor Growth Inhibition (TGI) of 0% indicates that mean tumor growth of the group was comparable to the mean tumor growth observed in the no treatment group, while 100% TGI indicates no tumor growth was observed between Day 5 to Day 20. A TGI >100% indicates that the mean tumor burden regressed from Day 5 to Day 20, while a TGI <0 indicates a tumor burden increase greater than the increase seen in the no treatment group (G1).

Treatment of mice with anti-CD19 CAR T cells prepared by the 3-day process or anti-CD19 CAR T cells prepared by the 7-day process resulted in prolonged survival compared with control mice that received NTD or no treatment. NTD controls and mice that received no treatment exited the study by Day 8 based on the survival setpoint defined by a tumor burden <5×10⁸ photons/second bioluminescence. By contrast, groups treated with anti-CD19 CAR T cells prepared by the 3-day process cells experienced significantly greater survival, with the mice from all dose cohorts achieving 100% survival at the study end point on Day 64. Mice treated with anti-CD19 CART cells prepared by the 7-day process had lower survival in comparison to groups treated with anti-CD19 CAR T cells prepared by the 3-day process, ranging from 80% survival for the 1×10⁷ CAR⁺ T-cell dose cohort to 20% survival for the 5×10 6 CAR′ T-cell dose cohort and 0% survival for the 3×10⁶ and 1×10⁶ CAR′ T-cell dose cohorts.

Mice that received anti-CD19 CAR T cells prepared by the 3-day process at all doses achieved a statistically significant improvement in response relative to those which received the anti-CD19 CAR T cells prepared by the 7-day process at the 3×10⁶ and 1×10⁶ CAR′ T-cell doses, but were not statistically different from the 1×10⁷ CAR′ T-cell dose of anti-CD19 CAR T cells prepared by the 7-day process. All doses of anti-CD19 CAR T cells prepared by the 3-day process resulted in complete anti-tumor responses and 100% survival by the end of study (Day 64), while the highest dose of anti-CD19 CAR T cells prepared by the 7-day process (1×10⁷ CAR⁺ T cells) resulted in an end-of-study survival of 80%.

Lastly, increasing tumor burden over time in the no treatment group led to a mean body weight loss of >18% by Day 26 and/or adverse clinical signs, after which all animals were removed from the study. Animals receiving NTD T cells showed a similar pattern of weight loss due to uncontrolled tumor burden by Day 29. In contrast, all doses of anti-CD19 CAR T cells prepared by the 3-day process from 1×10⁶ to 1×10⁵ cells were well tolerated, with animals maintaining a consistent body weight for the duration of the study. For groups treated with anti-CD19 CAR T cells prepared by the 7-day process, all mice at all doses tested (1×10⁷, 5×10⁶, 3×10⁶, and 1×10⁶ CAR⁺ T cells), maintained a consistent body weight for the duration of the study.

### Example 7 Characterization of Cells Prepared by a Three Day Process

This example describes functional and phenotypic characterization of anti-CD19/CD20 CAR T-cells that were harvested on day 3 as compared to anti-CD19/CD20 CAR T-cells that were harvested on day 6.

Briefly, positively selected CD4+ and CD8+ T cells from a healthy human donor were activated and transduced using a lentiviral vector encoding a dual targeting anti-CD19 CAR and anti-CD20 CAR. The transduced cells were subsequently harvested on Day 3. Similarly, another set of anti-CD19/CD20 CAR T-cell products were manufactured from T cells from the same donor but harvested on Day 6 (referred to in Examples 7 and 8 as “anti-CD19/CD20 CAR Day 6”). Subsequently, both CAR T-cell products were functionally characterized and compared with their respective NTD control T cells (NTD Day 3 and NTD Day 6) that were manufactured in parallel from the same donor material.

The anti-CD19/CD20 CAR T-cells that were harvested on day 3 were produced by the following 3-day process beginning with apheresis material collection. The starting apheresis material may optionally be fresh or cryopreserved apheresis or cryopreserved T cells. Wash 1, wash 2, T-cell enrichment, and wash 3 steps were conducted on day 0. T cell activation was conducted on day 0 up to day 1. Lentiviral transduction was conducted on day 1 up to day 4. Harvest wash and concentrate was conducted on day 3 up to day 4.

The anti-CD19/CD20 CAR T-cells that were harvested on day 6 were produced by the following process beginning with apheresis material collection. Wash 1, wash 2, T-cell enrichment, and wash 3 steps were conducted on day 0. T cell activation was conducted on day 0 up to day 2. Lentiviral transduction was conducted on day 2 up to day 5. Wash 4 was conducted on day 4 up to day 5. T-cell expansion was conducted on day 4 up to day 15. Harvest wash and concentrate was conducted on day 6 up to day 15.

The experiment groups for the studies described in this Example are shown in Table 17.

TABLE 17
Experiment Groups

| Group | Primary cells |
|---|---|
| 1 | NTD D 3 |
| 2 | anti-CD19/CD20 CAR D 3 |
| 3 | NTD D 6 |
| 4 | anti-CD19/CD20 CAR D 6 |

The anti-CD19/CD20 CAR T-cells produced by the 3-day process and the anti-CD19/CD20 CAR Day 6 T-cell products were assessed for CAR cell-surface expression to establish CAR transduction efficiencies. Cell-surface expression of both the anti-CD19 CAR and anti-CD20 CAR were individually detected by flow cytometry using fluorescent-labelled anti-idiotypic antibodies. Individual expression of the anti-CD19 CAR was 68% for anti-CD19/CD20 CAR T-cells produced by the 3-day process and 35% for the anti-CD19/CD20 CAR Day 6 T-cell product, and expression of the antiCD20 CAR was 65% and 37%, respectively, in the same samples. Transduction efficiency, as measured by the sum of total anti-CD19 and antiCD20 CAR expression (i.e., percentage of anti-CD19 CAR antibody+ cells plus percentage of anti-CD29 CAR antibody cells), was 72% for anti-CD19/CD20 CAR T-cells produced by the 3-day process and 40% for the antiCD19/CD20 CAR Day 6 T-cell product.

Phenotypic characterization of CAR T-cell products was performed by flow cytometry after incubation with fluorescent-labelled antibodies to assess the presence of CD4+ and CD8+ cells, and the relative composition of less differentiated T-cell subsets. CD45RA and CCR7 markers may be used to define the various populations of T cells, including naïve and stem memory T cells (CD45RA⁺CCR7⁺), also referred to as juvenile T cells. The anti-CD19/CD20 CAR T-cells produced by the 3-day process showed a slightly higher CD4/CD8 ratio than the anti-CD19/CD20 CAR Day 6 T-cell product (Table 18).

TABLE 18
CD4/CD8 ratio (% of CD3)

| | CD4 | CD8 |
|---|---|---|
| G1 | 55.8 | 41.4 |
| G2 | 53.8 | 42.1 |
| G3 | 51.9 | 47.1 |
| G4 | 49.2 | 48.6 |

Both CD4+ and CD8+ populations of anti-CD19/CD20 CAR T-cells produced by the 3-day process showed an increased frequency of juvenile cells compared with the anti-CD19/CD20 CAR Day 6 T-cell product (Table 19 and Table 20).

TABLE 19
CD45RA⁺CCR7⁺ (% of CD4)

| G1 | G2 | G3 | G4 |
|---|---|---|---|
| 67.3 | 71.7 | 31.3 | 22.8 |

TABLE 20
CD45RA⁺CCR7⁺ (% of CD8)

| G1 | G2 | G3 | G4 |
|---|---|---|---|
| 82.6 | 83.4 | 52.1 | 42.6 |

The functionality of the CAR T-cell products was assessed in co-culture assays with antigen-positive and antigen-negative target cells. NTD T-cell products were included in the experiments as controls to assess levels of alloreactivity. Cytotoxicity was measured at 1 day and 4 days after initiation of co-culture of the T-cell products with luciferase-expressing target cells. Cytotoxicity was measured as the reduction of the luciferase signal emitted in the test wells upon addition of luciferin compared with the wells where target cells alone were seeded. Multiple E:T ratios were used in these assays ranging from 1:1 to 1:243. Four main cell lines expressing various levels of CD19 and CD20 antigen were used: Raji, a B-cell lymphoma cell line that expresses high levels of CD19 and CD20 antigens; Nalm6, a B-cell leukemia cell line that expresses high levels of CD19 but low levels of CD20; ST486, a B-cell lymphoma cell line that expresses low levels of CD19 but high levels of CD20; and K-562, a chronic myelogenous leukemia cell line that does not express CD19 or CD20 antigens. In addition, controls consisted of Raji CD19 knockout (KO) cells, and Raji CD20KO cells. Raji cell lines were engineered to express either antigen: Raji CD19KO cells only express CD20 and not CD19, Raji CD20KO only express CD19 and not CD20. Antigen KO was confirmed at the DNA level by tracking of insertions or deletions (indels) by decomposition (TIDE) analysis and at the cell-surface protein level by flow cytometry.

At Day 4 of co-culture, of anti-CD19/CD20 CAR T-cells produced by the 3-day process and anti-CD19/CD20 CAR Day 6 T-cell products showed comparable dose-dependent specific cytotoxicity against antigen-positive target cells, regardless of expression of both or either antigen (Tables 21A-21F). Basal levels of antigen-independent cytotoxicity mediated by alloreactivity against target cells was observed by cytotoxicity of NTD D3 control T cells on ST486 cells (low CD19/high CD20 expression) at the highest E:T ratio. (Table 21A). Both of anti-CD19/CD20 CAR T-cells produced by the 3-day process and anti-CD19/CD20 CAR D6 T-cell products derived from a healthy donor had comparable cytotoxic activity across the various target cell lines tested and lacked killing activity of antigen-negative K-562 cells (Table 21F).

TABLE 21A
ST488 cells (CD19+CD20+) (% cytotoxicity)

| ET_Ratio | G2 | | | G4 | | | G1 | | | G3 | | |
|---|---|---|---|---|---|---|---|---|---|---|---|---|
| 0.0041 | 0 | 0 | 0 | 0 | 0 | 0 | 0 | 0 | 0 | 0 | 0 | 0 |
| 0.012 | 0 | 0 | 0 | 0 | 0 | 0 | 0 | 0 | 0 | 0 | 0 | 0 |
| 0.037 | 34.93 | 31.91 | 22.59 | 30.28 | 27.98 | 21.04 | 0 | 0 | 0 | 0 | 0 | 0 |
| 0.11 | 87.23 | 84.35 | 83.47 | 73.04 | 64.55 | 64.67 | 0 | 0 | 0 | 0 | 0 | 0 |
| 0.33 | 99.11 | 98.74 | 98.51 | 95.66 | 96.45 | 98.11 | 8.16 | 7.59 | 0.28 | 0 | 0 | 0 |
| 1 | 99.79 | 99.65 | 99.65 | 99.73 | 99.76 | 99.69 | 61.53 | 54.83 | 55.11 | 0 | 0 | 0 |

TABLE 21B
Nalm6 cells (CD19+CD20+) (% cytotoxicity)

| ET_Ratio | G2 | | | G4 | | | G1 | | | G3 | | |
|---|---|---|---|---|---|---|---|---|---|---|---|---|
| 0.0041 | 0 | 0 | 0 | 0 | 0 | 0 | 0 | 0 | 0 | 0 | 0 | 0 |
| 0.012 | 0 | 0 | 0 | 0 | 0 | 0 | 0 | 0 | 0 | 0 | 0 | 0 |
| 0.037 | 0 | 0 | 0 | 0 | 0 | 0 | 0 | 0 | 0 | 0 | 0 | 0 |
| 0.11 | 60.09 | 62.71 | 66.74 | 26.08 | 31.46 | 29.1 | 0 | 0 | 0 | 0 | 0 | 0 |
| 0.33 | 99.01 | 98.95 | 98.45 | 91.1 | 91.74 | 92.73 | 0 | 0 | 0 | 0 | 0 | 0 |
| 1 | 99.94 | 99.92 | 99.95 | 99.8 | 99.64 | 99.78 | 0 | 0 | 0 | 0 | 0 | 0 |

TABLE 21C
Raji cells (CD19+CD20+) (% cytotoxicity)

| ET_Ratio | G2 | | | G4 | | | G1 | | | G3 | | |
|---|---|---|---|---|---|---|---|---|---|---|---|---|
| 0.0041 | 0.67 | 1.85 | 0 | 0 | 0 | 0 | 0 | 0 | 0 | 0 | 0 | 0 |
| 0.012 | 0 | 0 | 0 | 0 | 0 | 0 | 0 | 0 | 0 | 0 | 0 | 0 |
| 0.037 | 0 | 0 | 0 | 0 | 0 | 3.12 | 0 | 0 | 0 | 0 | 0 | 0 |
| 0.11 | 6.2 | 7.04 | 2.67 | 6.29 | 5.23 | 3.01 | 0 | 0 | 0 | 0 | 0 | 0 |
| 0.33 | 23.82 | 23.4 | 21.59 | 22.3 | 23.09 | 22.26 | 0 | 0 | 0 | 0 | 0 | 0 |
| 1 | 55.98 | 54.26 | 53.38 | 53.83 | 53.61 | 53.91 | 8.96 | 4.07 | 2.58 | 8.63 | 6.24 | 3.13 |

TABLE 21D
Raji CD19KO cells (CD19−CD20+) (% cytotoxicity)

| ET_Ratio | G2 | | | G4 | | | G1 | | | G3 | | |
|---|---|---|---|---|---|---|---|---|---|---|---|---|
| 0.0041 | 0 | 0 | 0 | 0 | 0 | 0 | 0 | 0 | 0 | 0 | 0 | 0 |
| 0.012 | 1.97 | 0.02 | 5.07 | 0 | 0 | 3.64 | 0 | 0 | 0 | 0 | 0 | 0 |
| 0.037 | 4.02 | 12.24 | 3.54 | 3.4 | 11.08 | 4.41 | 0 | 0 | 0 | 0 | 0 | 0 |
| 0.11 | 7.88 | 11.55 | 10.77 | 9.01 | 11.11 | 17.12 | 0 | 0 | 0 | 0 | 0 | 0 |
| 0.33 | 27.08 | 34.69 | 27.04 | 21.47 | 31.31 | 33.68 | 0 | 0 | 0 | 0 | 0 | 0 |
| 1 | 51.83 | 52.51 | 49.39 | 45.94 | 52.37 | 49.69 | 0.3 | 12.75 | 5.14 | 0 | 1.79 | 0 |

TABLE 21E
Raji CD20KO cells (CD19+CD20−) (% cytotoxicity)

| ET_Ratio | G2 | | | G4 | | | G1 | | | G3 | | |
|---|---|---|---|---|---|---|---|---|---|---|---|---|
| 0.0041 | 2.13 | 5.08 | 6.03 | 10.71 | 7.73 | 6.1 | 0 | 0 | 0 | 0 | 1.58 | 0 |
| 0.012 | 17.55 | 16.79 | 16.18 | 24.96 | 19.97 | 18.29 | 8.54 | 5.02 | 14.91 | 7.75 | 7.88 | 5.99 |
| 0.037 | 31.28 | 29.9 | 29.89 | 28.39 | 28.21 | 27.88 | 11.98 | 11.38 | 9.98 | 14.52 | 12.93 | 8.95 |
| 0.11 | 30.11 | 20.83 | 32.51 | 29.55 | 29.13 | 30.22 | 1.54 | 0 | 0.5 | 7.33 | 2.56 | 6.88 |
| 0.33 | 24.09 | 24.84 | 24.58 | 37.77 | 34.9 | 37.91 | 0 | 0 | 4.72 | 1.67 | 0 | 1.78 |
| 1 | 51.67 | 47.7 | 48.05 | 47.12 | 48.77 | 45.02 | 8.09 | 11.02 | 11.07 | 3.49 | 8.43 | 7.68 |

TABLE 21F
K-562 cells (CD19−CD20−) (% cytotoxicity)

| ET_Ratio | G2 | | | G4 | | | G1 | | | G3 | | |
|---|---|---|---|---|---|---|---|---|---|---|---|---|
| 0.0041 | 0 | 0 | 0 | 0 | 0 | 0 | 0 | 0 | 0 | 0 | 0 | 0 |
| 0.012 | 0 | 0 | 0 | 0 | 0 | 0 | 0 | 0 | 0 | 0 | 0 | 0 |
| 0.037 | 0 | 0 | 0 | 0 | 0 | 0 | 0 | 0 | 0 | 0 | 0 | 0 |
| 0.11 | 0 | 0 | 0 | 0 | 0 | 0 | 0 | 0 | 0 | 0 | 0 | 0 |
| 0.33 | 0 | 0 | 0 | 0 | 0 | 0 | 0 | 0 | 0 | 0 | 0 | 0 |
| 1 | 0 | 0 | 3.37 | 0 | 0 | 1.59 | 0 | 0 | 0 | 0 | 0 | 0 |

Specific activity was further evaluated by measurement of cytokine levels in culture supernatants after an overnight co-culture of T-cell products with Nalm6, ST486, Raji, Raji CD19KO, Raji CD20KO, and K-562 target cells at an E:T ratio of 1:1. Quantitation of IFN-γ, IL-2, tumor necrosis factor-α (TNF-α) pro-inflammatory cytokine production was performed.

anti-CD19/CD20 CAR T-cells produced by a three-day process and anti-CD19/CD20 CAR Day 6 T-cell products showed robust antigen-dependent IFN-γ, IL-2, and TNF-α pro-inflammatory cytokine production from co-culture with the antigen-positive target cells, Nalm6, ST486, Raji, Raji CD19KO, and Raji CD20KO (Table 22A-22C). Both anti-CD19/CD20 CAR T-cells produced by a three-day process and anti-CD19/CD20 CAR Day 6 T-cell products showed comparable cytokine levels of IL-2 and TNF-α, across the various target cell lines tested. The functionality of each individual CAR was demonstrated by the production of cytokines when the T-cell products were co-cultured with target cells expressing a single antigen (Raji CD19KO or Raji CD20KO), albeit at lower levels than that induced by co-culture with Raji parental cells, which express both antigens. In contrast, no cytokine production was detected in the absence of target cells (i.e., T cells alone) and there was no cytokine production from CART cells co-cultured with antigen-negative K-562 (CD19−CD20−) cells as shown (Table 22A-22C)

TABLE 22A
IFN γ secretion (pg/mL)

| | G1 | | | G2 | | |
|---|---|---|---|---|---|---|
| Raji | 631.842023 | 673.719917 | 693.715378 | 88686.8936 | 88390.2695 | 95705.4359 |
| Raji CD19KO | 492.120724 | 546.261438 | 615.66846 | 56910.7259 | 61177.0689 | 64253.8024 |
| Raji CD20KO | 513.960957 | 762.309569 | 463.0381 | 80535.1897 | 85534.1247 | 73591.69 |
| Nalm6 | 556.3588 | 634.7961 | 846.9493 | 73772.59 | 71733.16 | 83234.62 |
| ST486 | 859.1064 | 739.7526 | 610.4024 | 102770.7 | 74682.97 | 84363.41 |
| K-562 | 504.6792 | 427.0096 | 494.3015 | 5068.977 | 5101.474 | 4395.067 |
| T Cells alone | 493.22016 | 439.41505 | 560.89536 | 3804.1729 | 2774.8777 | 3279.106 |

| | G3 | | | G4 | | |
|---|---|---|---|---|---|---|
| Raji | 64.633201 | 85.182547 | 62.767611 | 72703.3938 | 62697.5733 | 73943.3727 |
| Raji CD19KO | 50.653482 | 32.994943 | 46.00049 | 45363.4252 | 43389.0288 | 45471.0076 |
| Raji CD20KO | 59.970116 | 40.422233 | 58.25347 | 52114.3441 | 54101.4732 | 56705.47 |
| Nalm6 | 24.02465 | 32.34113 | 21.25442 | 69690.74 | 74599.62 | 72071.31 |
| ST486 | 54.5489 | | | 81068.87 | 59904.5 | 66741.56 |
| K-562 | 112.971583 | 14.7083822 | 10.3012139 | | | |
| T Cells alone | 15.963588 | 20.0692 | 16.993131 | 61.042722 | 59.292719 | 65.083158 |

TABLE 22B
IL-2 secretion (pg/mL)

| | G1 | | | G2 | | |
|---|---|---|---|---|---|---|
| Raji | | 1.2715598 | 1.9489383 | 4272.88282 | 5156.01144 | 4736.15664 |
| Raji CD19KO | 5.6393266 | 0.9313962 | 1.9489383 | 907.473679 | 944.985293 | 1060.583 |
| Raji CD20KO | 0.2456634 | 4.3021465 | 2.121362 | 4067.82741 | 3431.4832 | 3784.99 |
| Nalm6 | | | | 3087.476 | 3506.963 | 3593.429 |
| ST486 | | 0.02115656 | 10.4754167 | 4327.265 | 3690.72 | 3809.59 |
| K-562 | 0 | 0 | 0 | 6.46259237 | 5.92807962 | 8.63028699 |
| T Cells alone | 1.5061941 | 2.0514315 | 1.6878284 | 7.6561311 | 4.574488 | 4.848683 |
| | G3 | | | G4 | | |
| Raji | 4.9712569 | 13.273517 | 3.2961473 | 3020.23907 | 3257.4112 | 3424.93298 |
| Raji CD19KO | 5.6393266 | | 2.2865335 | 936.790753 | 927.019416 | 1056.80524 |
| Raji CD20KO | | | 5.047304 | 1720.39506 | 1631.67487 | 1711.666 |
| Nalm6 | 4.722206 | | | 1930.181 | 2080.665 | 2074.802 |
| ST486 | | | | 2868.025 | 2615.041 | 2629.092 |
| K-562 | 0 | 0.40768438 | 0.06331794 | 1.39388945 | 1.59690516 | 3.24797964 |
| T Cells alone | 0.7510095 | 2.2637136 | 1.5969961 | 3.3267079 | 3.2659041 | 3.9959589 |

TABLE 22C
TNF α secretion (pg/mL)

| | G1 | | | G2 | | |
|---|---|---|---|---|---|---|
| Raji | 23.813341 | 19.161838 | 20.192636 | 754.338361 | 891.841792 | 751.149696 |
| Raji CD19KO | 15.057532 | 13.018343 | 16.336655 | 357.632019 | 344.056688 | 461.368626 |
| Raji CD20KO | 22.518038 | 8.7215178 | 6.306 | 929.466749 | 898.546424 | 844.7008 |
| Nalm6 | | | | 821.6964 | 773.5773 | 844.172 |
| ST486 | 1.608184 | 19.8814103 | 21.0210015 | 878.2847 | 793.4574 | 897.9296 |
| K-562 | 1.47838933 | 0.47430105 | 0 | 6.70118515 | 5.88777721 | 6.81731438 |
| T Cells alone | 1.8959588 | 1.6426018 | 2.7219428 | 12.654293 | 10.497499 | 14.290262 |
| | G3 | | | G4 | | |
| Raji | 5.9760712 | 8.7215178 | 4.9869821 | 598.931052 | 475.071069 | 689.204856 |
| Raji CD19KO | 3.7596826 | 12.256219 | 10.484037 | 279.821783 | 271.683547 | 347.448925 |
| Raji CD20KO | 8.4706165 | 3.2721687 | 1.08748 | 524.274078 | 594.040007 | 527.0265 |
| Nalm6 | | | | 587.2551 | 614.7329 | 578.2727 |
| ST486 | | 7.07694449 | 12.7634852 | 672.34 | 647.3185 | 609.3595 |
| K-562 | 0.17620014 | 0 | 0 | 1.27235707 | 1.15449786 | 0.71149013 |
| T Cells alone | 1.168786 | 1.1321249 | 2.9004278 | 1.3150979 | 1.8598301 | 2.9717291 |

To assess antigen-specific proliferation of the CAR T-cell products upon engagement with target antigens, T-cell products that had been co-cultured with Raji, Nalm6, Raji CD19KO, Raji CD20KO, and K-562 target cells were harvested 4 days after initiation of co-culture, centrifuged to pellets, and prepared for flow cytometry evaluation. T cells had been prelabeled with a fluorescent dye used to trace multiple generations of cells by analysis of dye dilution upon cell division. The 1:1 E:T ratio from the co-cultures was used and the T-cell products that had been cultured in the absence of target cells (T cells alone) were used as controls. T cells co-cultured with antigen-negative K-562 (CD19⁻ CD20⁻) cells were used to assess specificity and levels of antigen-independent proliferation while T cells alone were used to assess basal levels of proliferation in the absence of stimuli. Upon harvest, cells were stained with fluorescent-labeled antibodies against CD3, CD4, CD8, CD19 CAR, CD20 CAR, CD25 and a viability dye.

Anti-CD19/CD20 CAR T-cells produced by a three-day process and anti-CD19/CD20 CAR Day 6 T-cell products showed comparable proliferation in co-culture with Nalm6, Raji, Raji CD19KO, and Raji CD20KO target cells, but not with antigen-negative K-562 cells, demonstrating that both the anti-CD19 CAR and anti-CD20 CAR in the T-cell products are functional. The NTD T-cell controls showed various levels of nonspecific basal proliferation when co-cultured with the different target cell lines, but overall, the NTD Day 3 cells demonstrated more basal proliferation than the NTD Day 6 cells.

### Example 8 In Vivo Efficacy of Cells Prepared by a Three-Day Process

In vivo studies were conducted in a disseminated xenograft mouse model of human B-ALL, which consists of profoundly immunodeficient nonobese diabetic (NOD), severe combined immunodeficiency (scid) IL-2 receptor-gamma chain null (NSG) mice injected intravenously with the Nalm6-luc cells, expressing high levels of CD19 and CD20. Antitumor efficacy was assessed for CAR T-cell products that were generated from T cells derived from 1 healthy donor and previously characterized by in vitro studies as described in Example 7.

Nalm6luc cells (5.0×10⁵) were implanted intravenously via the lateral tail vein into 7 week-old female NSG mice (5 mice per group). On Day 6 after tumor implantation, mice received either controls (vehicle [phosphate-buffered saline] or NTD Day 3 cells) or anti-CD19/CD20 CAR T cells, as follows: Anti-CD19/CD20 CAR T-cells produced by a three-day process at 1 of 3 dose levels (2.0×10⁵, 4.0×10⁴, and 8.0×10³ CAR⁺ T cells); antiCD19/CD20 CAR Day 6 T-cell product at 1 dose level (2.0×10⁵ CAR⁺ T cells). All animals were dosed with a single IV administration of applicable treatment at a fixed volume of 100 μL and mice from the vehicle group received an IV injection of 100 μL PBS. Table 23 shows the experimental groups for the results represented in Tables 24-26.

TABLE 23
Experiment Groups for Results presented in Tables 24-26

| Group | N | Primary cells | CAR+ dose/kg |
|---|---|---|---|
| G1 | 5 | Vehicle | N/A |
| G2 | 5 | NTD_D 3 | 5.0 × 10⁵ (T cells) |
| G3 | 5 | Anti-CD19/CD20 CAR_D 3 | 2.0 × 10⁵ |
| G4 | 5 | Anti-CD19/CD20 CAR_D 3 | 4.0 × 10⁴ |
| G5 | 5 | Anti-CD19/CD20 CAR_D 3 | 8.0 × 10³ |
| G6 | 5 | Anti-CD19/CD20 CAR_D 6 | 2.0 × 10⁵ |

Antitumor activity was evaluated by assessing the change in tumor burden (measured by bioluminescence and log₁₀ normalized) from Day 5 (1 day before CAR T-cell infusion) to Day 22 (i.e., 16 days after CAR T-cell infusion, and the last time point at which all groups were intact). Treatment with anti-CD19/CD20 CAR T-cells produced by a three-day process resulted in a significant reduction in tumor burden when compared with mice that received either vehicle (PBS) or NTD Day 3 T cells (Table 24).

TABLE 24
Antitumor Activity of T-cell Products on the Tumor
Burden of Nalm6-luc -bearing NSG Mice (photons/s)

| Days | G1 | | | | |
|---|---|---|---|---|---|
| 5 | 1.27E+07 | 6.11E+06 | 6.06E+06 | 4.56E+06 | 4.53E+06 |
| 8 | 1.28E+08 | 5.72E+07 | 6.38E+07 | 4.31E+07 | 6.22E+07 |
| 12 | 5.42E+09 | 4.00E+09 | 2.38E+09 | 2.60E+09 | 2.13E+09 |
| 15 | 1.39E+10 | 8.68E+09 | 9.48E+09 | 9.49E+09 | 9.04E+09 |
| 19 | 2.23E+10 | 2.80E+10 | 8.25E+08 | 2.86E+10 | 2.57E+10 |
| 22 | 4.47E+10 | 4.17E+10 | 4.59E+10 | 3.55E+10 | 3.92E+10 |
| Days | G2 | | | | |
| 5 | 1.23E+07 | 6.14E+06 | 5.75E+06 | 4.71E+06 | 4.50E+06 |
| 8 | 1.30E+08 | 5.09E+06 | 5.66E+07 | 5.70E+07 | 4.59E+07 |
| 12 | 6.18E+09 | 3.42E+09 | 3.20E+09 | 2.99E+09 | 2.62E+09 |
| 15 | 1.56E+10 | 1.35E+10 | 1.57E+10 | 1.60E+10 | 1.08E+10 |
| 19 | 3.26E+10 | 2.86E+10 | 3.02E+10 | 3.48E+10 | 3.27E+10 |
| 22 | 4.75E+10 | 4.82E+10 | 5.80E+10 | 6.61E+10 | 4.67E+10 |
| Days | G3 | | | | |
| 5 | 8.26E+06 | 6.56E+06 | 5.62E+06 | 4.84E+06 | 4.09E+06 |
| 8 | 9.42E+07 | 7.78E+07 | 7.66E+07 | 8.78E+07 | 6.56E+07 |
| 12 | 1.18E+08 | 1.97E+08 | 1.52E+08 | 1.50E+08 | 2.18E+08 |
| 15 | 4.28E+06 | 4.36E+06 | 1.10E+07 | 5.90E+06 | 1.67E+07 |
| 19 | 7.96E+05 | 9.64E+05 | 9.66E+05 | 7.64E+05 | 9.74E+05 |
| 22 | 6.41E+05 | 6.28E+05 | 7.37E+05 | 6.58E+05 | 7.24E+05 |
| 26 | 6.10E+05 | 7.45E+05 | 7.47E+05 | 6.72E+05 | 7.17E+05 |
| 29 | 6.42E+05 | 7.91E+05 | 1.19E+06 | 8.19E+05 | 8.23E+05 |
| 33 | 6.40E+05 | 7.79E+05 | 1.24E+06 | 9.26E+05 | 9.22E+05 |
| 36 | 6.50E+05 | 8.02E+05 | 1.43E+06 | 9.25E+05 | 8.02E+05 |
| 40 | 6.77E+05 | 7.81E+05 | 1.72E+06 | 8.67E+05 | 9.19E+05 |
| 44 | 8.32E+05 | 8.66E+05 | 3.04E+06 | 9.40E+05 | 1.09E+06 |
| 48 | 7.81E+05 | 8.16E+05 | 2.33E+06 | 7.85E+05 | 9.18E+05 |
| 51 | 8.04E+05 | 1.27E+06 | 6.47E+07 | 3.49E+06 | 9.71E+05 |
| 55 | 1.72E+06 | 4.65E+06 | 4.89E+08 | 1.60E+07 | 1.90E+06 |
| 58 | 1.20E+06 | 3.62E+06 | 3.83E+08 | 6.81E+06 | 1.13E+06 |
| 61 | 1.60E+07 | 1.31E+08 | 8.78E+09 | 2.15E+08 | 1.48E+07 |
| 65 | 3.97E+07 | 4.56E+08 | 1.20E+10 | 3.29E+08 | 2.92E+07 |
| Days | G4 | | | | |
| 5 | 8.14E+06 | 6.59E+06 | 5.60E+06 | 5.08E+06 | 3.67E+06 |
| 8 | 4.50E+06 | 6.44E+07 | 8.50E+07 | 5.81E+07 | 4.03E+06 |
| 12 | 2.33E+09 | 2.40E+09 | 1.86E+09 | 1.59E+09 | 1.18E+09 |
| 15 | 3.07E+09 | 7.27E+09 | 3.02E+09 | 2.10E+09 | 1.48E+09 |
| 19 | 1.02E+06 | 1.14E+06 | 8.76E+05 | 1.07E+06 | 8.67E+05 |
| 22 | 8.96E+05 | 9.88E+05 | 1.02E+06 | 8.63E+05 | 6.80E+05 |
| 26 | 6.46E+05 | 9.76E+05 | 7.79E+05 | 6.63E+05 | 7.98E+05 |
| 29 | 6.71E+05 | 5.74E+05 | 6.93E+05 | 6.35E+05 | 6.12E+05 |
| 33 | 8.45E+05 | 1.01E+06 | 8.17E+05 | 9.12E+05 | 8.15E+05 |
| 36 | 8.75E+05 | 7.00E+05 | 1.01E+06 | 8.68E+05 | 9.22E+05 |
| 40 | 7.64E+05 | 1.35E+06 | 1.02E+06 | 8.80E+05 | 9.03E+05 |
| 44 | 9.17E+05 | 1.30E+06 | 1.07E+06 | 1.04E+06 | 9.77E+05 |
| 48 | 8.42E+05 | 3.24E+06 | 1.12E+06 | 1.88E+06 | 9.78E+05 |
| 51 | 9.25E+05 | 9.93E+05 | 1.14E+06 | 3.96E+06 | 1.26E+06 |
| 55 | 8.83E+05 | 5.95E+06 | 1.17E+06 | 6.44E+05 | 7.58E+05 |
| 58 | 1.05E+06 | 3.07E+06 | 1.38E+06 | 1.38E+06 | 7.95E+05 |
| 61 | 2.14E+06 | 1.14E+08 | 5.43E+06 | 4.49E+06 | 1.38E+06 |
| 65 | 1.19E+07 | 1.05E+09 | 2.37E+07 | 1.94E+07 | 2.26E+06 |
| Days | G5 | | | | |
| 5 | 7.75E+06 | 6.60E+06 | 5.54E+06 | 5.14E+06 | 3.44E+06 |
| 8 | 3.90E+06 | 7.80E+07 | 7.16E+07 | 5.36E+07 | 2.84E+06 |
| 12 | 4.03E+09 | 3.73E+09 | 2.73E+09 | 2.22E+09 | 4.23E+09 |
| 15 | 1.41E+10 | 9.71E+09 | 1.04E+10 | 7.87E+09 | 1.30E+10 |
| 19 | 2.18E+10 | 1.99E+10 | 2.18E+10 | 1.64E+10 | 2.03E+10 |
| 22 | 2.51E+10 | 2.78E+10 | 3.45E+10 | 1.24E+10 | 1.84E+10 |
| 26 | 2.21E+10 | 4.28E+10 | 4.11E+10 | 2.01E+10 | 1.27E+10 |
| 29 | 3.01E+10 | 4.71E+10 | 8.67E+10 | 2.14E+10 | 9.90E+09 |
| 33 | | | | | 2.64E+10 |
| Days | G6 | | | | |
| 5 | 1.03E+07 | 6.46E+06 | 5.66E+06 | 4.81E+06 | 4.33E+06 |
| 8 | 8.05E+07 | 4.06E+07 | 3.95E+07 | 2.32E+07 | 1.42E+07 |
| 12 | 3.73E+07 | 1.94E+06 | 2.90E+06 | 3.68E+06 | 1.52E+06 |
| 15 | 3.21E+06 | 7.48E+05 | 7.54E+05 | 8.15E+05 | 8.42E+05 |
| 19 | 1.44E+06 | 1.21E+06 | 1.45E+06 | 1.28E+06 | 1.20E+06 |
| 22 | 2.74E+06 | 1.40E+06 | 2.96E+06 | 2.14E+06 | 1.31E+06 |
| 26 | 8.18E+06 | 3.32E+06 | 1.49E+07 | 8.39E+06 | 4.55E+06 |
| 29 | 5.40E+07 | 2.11E+07 | 1.39E+08 | 6.41E+07 | 3.06E+07 |
| 33 | 1.38E+09 | 4.60E+08 | 4.35E+09 | 1.95E+09 | 1.32E+09 |
| 36 | 1.07E+10 | 4.52E+09 | 1.39E+10 | 8.28E+09 | 6.10E+09 |
| 40 | 2.92E+10 | 1.55E+10 | 2.61E+10 | 2.13E+10 | 1.81E+10 |
| 44 | 2.60E+10 | 3.51E+10 | 4.68E+10 | 4.37E+10 | 5.06E+10 |

Inhibition and regression of tumor growth were observed in the 2.0×10⁵ (124.3%) and 4.0×10⁴ (121.1%) CAR⁺ T-cell dose cohorts of anti-CD19/CD20 CAR T-cells produced by a three-day process; and at the lowest dose of 8.0×10³ CAR⁺ T cells, mice displayed a slight tumor growth inhibition (5.5%). Tumor growth was controlled in animals that received the 2.0×10⁵ CAR⁺ T-cell dose of anti-CD19/CD20 CAR Day 6 with a tumor growth inhibition of 112.5% when compared with the vehicle group (Table 24, Table 25).

TABLE 25
Log-transform Analysis of Tumor Burden

| | G1 | G2 | G3 | G4 | G5 | G6 |
|---|---|---|---|---|---|---|
| Day 5 | 6.80 | 6.79 | 6.76 | 6.75 | 6.74 | 6.78 |
| Day 22 | 10.62 | 10.72 | 5.83 | 5.94 | 10.35 | 6.30 |
| Change, Day 5 to Day 22 | 3.82 | 3.93 | −0.93 | −0.80 | 3.61 | −0.48 |
| ΔT/ΔC | 100.0% | 102.9% | −24.3% | −21.1% | 94.5% | −12.5% |
| Tumor Growth Inhibition, | 0.0% | −2.9% | 124.3% | 121.1% | 5.5% | 112.5% |
| Day 22 | | | | | | |

Tumor burden (measured by BLI) was login normalized, with means for each cohort at Day 5 and Day 22 of the study represented in Table 25. The change in tumor BLI from Day 5 to Day 22 was calculated, and the percentage change in tumor burden relative to the vehicle group was calculated as ΔT/ΔC. Tumor growth inhibition was defined as (1-[ΔT/ΔC]×100) and represented the percentage tumor volume change in study arms relative to the control (ie, mice that received vehicle [PBS]). A TGI of 0% indicates that the mean tumor growth of the group was comparable to the mean tumor growth observed in mice that received vehicle, while 100% TGI indicates no tumor growth was observed between Day 5 to Day 22. A TGI >100% indicates that the mean tumor burden regressed from Day 5 to Day 22, while a TGI <0 indicates a tumor burden increase greater than the increase seen in mice that received vehicle.

Antitumor efficacy, as determined by the analysis of tumor burden, reached statistical significance on Day 22 for the mice treated with anti-CD19/CD20 CAR T-cells produced by a three-day process at all doses tested (2.0×10⁵, 4.0×10⁴, and 8.0×10³ CAR⁺ T cells) and for the mice treated with the anti-CD19/CD20 CAR Day 6 product at 2.0×10⁵ CAR⁺ T cells (Table 26).

TABLE 26
Statistical Analysis of Tumor Burden, Day 22

| Treatment Groups | G1 | G2 | G3 | G4 | G5 | G6 |
|---|---|---|---|---|---|---|
| G1 | | ns | **** | **** | ** | **** |
| G2 | ns | | **** | **** | *** | **** |
| G3 | **** | **** | | ns | **** | **** |
| G4 | **** | **** | ns | | **** | *** |
| G5 | ** | *** | **** | **** | | **** |
| G6 | **** | **** | **** | *** | **** | |

In Table 26, Log₁₀ normalized tumor BLI data at Day 22 was assessed for statistical significance across all groups using analysis of variance with Tukey's post-test. Significance between comparisons is noted as follows: ns, P >0.05; **, P<0.01; ***, P<0.001; ****, P<0.0001.

Treatment of mice with anti-CD19/CD20 CAR T-cells produced by a three-day process or anti-CD19/CD20 CAR Day 6 T-cell products resulted in increased overall survival (OS) compared with mice that received vehicle or NTD controls. All mice that received either vehicle or NTD controls exited the study by Day 15 or Day 12, respectively, based on the survival setpoint defined by tumor burden <1×10¹⁰ photons/second bioluminescence. Groups treated with anti-CD19/CD20 CAR T-cells produced by a three-day process at the highest doses of 2.0×10⁵ and 4.0×10⁴ CAR⁺ T cells achieved 80% and 100% survival, respectively, at the study end point. Mice treated with the lowest dose of anti-CD19/CD20 CAR T-cells produced by a three-day process (8.0×10³ CAR⁺ T cells) reached a median survival of 12 days, similar to the mice from the control groups (vehicle and NTD Day 3). At the same dose of 2.0×10⁵ CAR⁺ T cells, mice that received anti-CD19/CD20 CAR Day 6 T-cell product had a reduced median survival (36 days) when compared with mice treated with anti-CD19/CD20 CAR T-cells produced by a three-day process (median survival point not achieved).

In summary, treatment with anti-CD19/CD20 CAR T-cells produced by a three-day process significantly delayed tumor growth and increased survival in a Nalm6-luc human B-ALL xenograft NSG mouse model.

### Example 9: Additional Phenotypic Characteristics of Cells Manufactured Using a 3-Day Process

This example complements Example 3 and presents additional data related to the phenotypic characteristics of the harvested cells manufactured using the 3-day process.

Tables 27 and 28 show final product data for cells prepared using the 3-day process. The cells were derived from healthy donor material (fresh apheresis or frozen PBMCs; N1-N9) and from patient material (frozen PBMCs; P1-P4). The final product runs exhibited higher frequencies of central memory (CM) and juvenile cells compared to Teff/TEMRA and effector memory (EM) cell populations, indicating a more juvenile product (Tables 27 and 28).

TABLE 27

| | CD3+ T cells (%) | | CD4+ T cells (%) | | | |
|---|---|---|---|---|---|---|
| | CD4 | CD8 | CM | Juvenile | Teff/TEMRA | EM |
| N1 | 46.7 | 49.2 | 37 | 56.3 | 0.29 | 6.39 |
| N2 | 49.7 | 46.8 | 27.3 | 63.8 | 0.89 | 8.02 |
| N3 | 32.4 | 54.1 | 37.9 | 53.5 | 0.6 | 8.0 |
| N4 | 66 | 30.1 | 44.8 | 42.9 | 0.5 | 11.8 |
| N5 | 55.7 | 41.4 | 52.9 | 46 | 0 | 1 |
| N6 | 65.1 | 31.1 | 37.2 | 52.8 | 2.2 | 7.9 |
| N7 | 72.9 | 23.6 | 56.8 | 27.2 | 0.2 | 15.9 |
| N8 | 36.3 | 59.8 | 58.8 | 23.8 | 0.6 | 16.8 |
| N9 | 53.5 | 42.8 | 24.7 | 66.3 | 2.2 | 6.8 |
| P1 | 81.6 | 15.1 | 58.4 | 27.3 | 3.4 | 10.9 |
| P2 | 70.8 | 27.2 | 53.3 | 34.3 | 0.6 | 11.8 |
| P3 | 39.5 | 42.2 | 84.7 | 6.2 | 0.6 | 8.5 |
| P4 | 71.2 | 25.6 | 84.3 | 4.1 | 0.3 | 11.3 |

TABLE 28

CD8+ T cells (%)

| | CM | Juvenile | Teff/TEMRA | EM |
|---|---|---|---|---|
| N1 | 67.9 | 25.6 | 0.36 | 6.16 |
| N2 | 62.2 | 27.3 | 2.62 | 7.9 |
| N3 | 65.1 | 19.7 | 0.7 | 14.5 |
| N4 | 54.6 | 33.1 | 0.7 | 11.5 |
| N5 | 62.5 | 31.8 | 3.5 | 2.3 |
| N6 | 70.5 | 9.3 | 4.4 | 15.8 |
| N7 | 62.1 | 19.1 | 0.7 | 18.2 |
| N8 | 81.5 | 10.6 | 0.3 | 7.6 |
| N9 | 63.0 | 28.3 | 0.7 | 7.9 |
| P1 | 71.2 | 3.2 | 0.8 | 24.8 |
| P2 | 45.4 | 12.9 | 0.5 | 41.2 |
| P3 | 93.3 | 0.4 | 0.3 | 6.0 |
| P4 | 65.7 | 3.6 | 11.6 | 19.0 |

In Tables 27 and 28, the abbreviations are as follow: CM, central memory; EM, effector memory; HD, healthy donor; TEMRA, terminally differentiated effector memory T cells. CD45RA and CCR7 were used to define T cell phenotype subsets, which include juvenile T cells (Naïve, CD45RA+CCR7+), CM (CD45RA−CCR7+), EM (CD45RA−CCR7−), and Teff/TEMRA (CD45RA+CCR7−)

As seen in Table 27, the percentage of CD4+ CCR7+ cells (i.e. CM and Juvenile cells) in the final harvested product is at least 80% of the harvested CD4+ T-cell population. Put another way, the maximum percentage of Teff/TEMRA and EM CD4+ cells in the final harvested population is about 20%. Thus, there are at least 4 times as many CD4+ CCR7+ cells in the harvested population as Teff/TEMRA and EM CD4+ cells.

As seen in Table 28, the percentage of CD8+ CCR7+ cells (i.e. CM and Juvenile cells) in the final harvested product is at least about 60% of the harvested CD8+ T-cell population. Put another way, the maximum percentage of Teff/TEMRA and EM CD8+ cells in the final harvested population is about 40%. Thus, there are at least 1.5 times as many CD8+ CCR7+ cells in the harvested population as Teff/TEMRA and EM CD8+ cells.

### Example 10: Characterization of Cells Preparedly a Three Day Process

This example describes functional and phenotypic characterization of anti-CLL-1 CAR T-cells that were harvested on day 3 as compared to anti-CLL-1 CAR T-cells that were harvested on day 8. The anti-CLL-1 CAR T cells described in this example were produced by a similar method as described in Example 7.

Briefly, T cells selected from a healthy human donor were activated on day 0 and transduced on day 1 using a lentiviral vector encoding an anti-CLL-1 CAR. The transduced cells were subsequently harvested on Day 3. Another set of anti-CLL-1 CAR T-cells were manufactured from T cells from the same donor but harvested on Day 8. Subsequently, both CAR T-cell products were functionally characterized and compared with their respective NTD control T cells (NTD Day 3 and NTD Day 8).

The experiment groups for the studies described in this Example are shown in Table 29.

TABLE 29
Experiment Groups

| Group | Primary cells |
|---|---|
| 1 | anti-CLL-1 CAR day 3 |
| 2 | NTD day 3 |
| 3 | anti-CLL-1 CAR day 8 |
| 4 | NTD day 8 |

The anti-CLL-1 CAR T cells harvested on day 3 and day 8 were assessed for CAR cell-surface expression to establish CAR transduction efficiencies. CAR expression was comparable between those harvested on day 3 and day 8. Expression of the anti-CLL-1 CAR was 60.4% for the anti-CLL-1 CAR T-cells harvested on day 3 and 78.9% for the anti-CLL-1 CAR T-cells harvested on day 8.

Phenotypic characterization of CAR T-cell products was performed by flow cytometry. The anti-CLL-1 CAR T cells harvested on day 3 showed an increased juvenile T cell profile with 83.7% CD45RA⁺CCR7⁺ cells on day 3 harvest compared to 23% CD45RA⁺CCR7⁺ on day 8 harvest.

Anti-CLL-1 CAR T cells harvested on day 3 displayed increased CD4+ cells compared to those harvested on day 8 as shown in Table 30.

TABLE 30
Percent of CD4+ and CD8+

| | CD4+ | CD8+ |
|---|---|---|
| G1 | 85.7 | 12.1 |
| G2 | 87.6 | 10.8 |
| G3 | 39 | 59.6 |
| G4 | 34.5 | 64.4 |

The functionality of the CAR T-cell products was assessed in co-culture assays with antigenpositive target cells. NTD T-cell products were included in the experiments as controls. Cytotoxicity was measured at 1 day and 4 days after initiation of coculture of the T-cell products. E:T ratios of 1:1 to 1:3 were used. The MV4-11 (CLL-1 medium expression) and Kasumi-1 (CLL-1 very low expression) cell lines were used.

Anti-CLL-1 CAR T-cells harvested on day 3 showed increased cytotoxicity in vitro against Kasumi-1 cells compared to anti-CLL-1 CAR t cells harvested on day 8 (Tables 30A-30B). Anti-CLL-1 CAR T-cells harvested on day 3 and day 8 showed comparable cytotoxicity in vitro against MV4-11 cells (Tables 30C-30D).

TABLE 30A
Kasumi-1 24 hr cytotoxicity (% cytotoxicity; n = 3)

| | G1 | | G3 | | G2 | | G4 | |
|---|---|---|---|---|---|---|---|---|
| ET_Ratio | Mean | SD | Mean | SD | Mean | SD | Mean | SD |
| 1:3 | 31.39755 | 7.237829 | 5.72886 | 0.190338 | 10.74357 | 10.65613 | −1.74482 | 1.001606 |
| 1:1 | 31.1249 | 8.885557 | 1.456459 | 2.737722 | 19.24212 | 14.33823 | −14.8815 | 2.310444 |

TABLE 30B
Kasumi-1 96 hr cytotoxicity (% cytotoxicity; n = 3)

| | G1 | | G3 | | G2 | | G4 | |
|---|---|---|---|---|---|---|---|---|
| ET_Ratio | Mean | SD | Mean | SD | Mean | SD | Mean | SD |
| 1:3 | 52.72296 | 5.543031 | 61.92385 | 2.352123 | 16.17232 | 3.449642 | 7.881773 | 1.533963 |
| 1:1 | 71.15299 | 3.609597 | | | 51.42474 | 4.706712 | 45.29149 | 1.63592 |

TABLE 30C
MV4-11 24 hr cytotoxicity (% cytotoxicity; n = 3)

| | G1 | | G3 | | G2 | | G4 | |
|---|---|---|---|---|---|---|---|---|
| ET_Ratio | Mean | SD | Mean | SD | Mean | SD | Mean | SD |
| 1:3 | 50.36574 | 13.43492 | 28.20413 | 0.961508 | 0.635403 | 5.109987 | −10.2269 | 1.117845 |
| 1:1 | 62.16195 | 4.45965 | 54.16878 | 0.382472 | 18.13723 | 12.05343 | −14.0123 | 1.843203 |

TABLE 30D
MV4-11 96 hr cytotoxicity (% cytotoxicity; n = 3)

| | G1 | | G3 | | G2 | | G4 | |
|---|---|---|---|---|---|---|---|---|
| ET_Ratio | Mean | SD | Mean | SD | Mean | SD | Mean | SD |
| 1:3 | 91.10783 | 3.76045 | | | 19.22778 | 1.837575 | 10.31388 | 1.704941 |
| 1:1 | 89.19031 | 3.246625 | 99.74822 | 0.031169 | 16.87958 | 0.044702 | 13.43041 | 2.969454 |

### Example 11: Further Characterization of Cells Prepared by a 3-Day Process

In this example the phenotypic characteristics of cells produced using a 3-day process similar to that described in Example 3 above are reported. In this example, a lentiviral vector was used to deliver the CAR.

First, as shown in Table 31, harvested cells generated from the 3-day process had a greater CD4/CD8 ratio as compared to cells harvested from the 7-day process. Table 31 discloses the values from 4 individual samples per identified group.

TABLE 31
Cells generated from the 3-day process had a greater
CD4/CD8 ratio as compared to cells harvested from
the 7-day process. (NTD = non-transduced control.)

| Group | Sample | CD4/CD8 ratio |
|---|---|---|
| 3-day NTD | 1 | 20.50438596 |
| controls | 2 | 8.466019417 |
| (n = 4) | 3 | 4.174863388 |
| | 4 | 6.453125 |
| 7-day NTD | 1 | 3.143459916 |
| controls | 2 | 1.809798271 |
| (n = 4) | 3 | 1.063965885 |
| | 4 | 2.210702341 |
| 3-day cells | 1 | 12.86956522 |
| (n = 4) | 2 | 7.016949153 |
| | 3 | 4.2 |
| | 4 | 6.373015873 |
| 7-day cells | 1 | 2.590405904 |
| (n = 4) | 2 | 2.105769231 |
| | 3 | 1.023255814 |
| | 4 | 2.022151899 |

Next, as shown in Table 32, harvested cells generated from the 3-day process had greater percentages of CD45RA+CCR7+ cells and CD45RA-CCR7− cells as compared to cells harvested from the 7-day process. Table 32 discloses the values from 4 individual samples per identified group. (NTD=non-transduced control.)

TABLE 32
CD45RA + CCR7+ cells and CD45RA − CCR7− cells are more
abundant in harvested cells from the 3-day process

| | | Avg. % of live CD3 T-cells | |
|---|---|---|---|
| | | CD45RA + CCR7+ | CD45RA − CCR7− |
| Group | Sample | cells | cells |
| 3-day NTD | 1 | 61.9 | 33.8 |
| control | 2 | 47.1 | 47.3 |
| (n = 4) | 3 | 69 | 2.45 |
| | 4 | 32.9 | 26.7 |
| 7-day NTD | 1 | 36.5 | 21.4 |
| control | 2 | 30.8 | 25.6 |
| (n = 4) | 3 | 17.8 | 0.19 |
| | 4 | 5.49 | 10.2 |
| 3-day cells | 1 | 58 | 34.3 |
| (n = 4) | 2 | 33.4 | 55.8 |
| | 3 | 70 | 3.15 |
| | 4 | 28.9 | 28 |
| 7-day cells | 1 | 26.9 | 24.8 |
| (n = 4) | 2 | 20 | 33.2 |
| | 3 | 16 | 0.12 |
| | 4 | 3.31 | 8.81 |

Next, as shown in Table 33, harvested cells generated from the 3-day process had greater percentages of Tscm cells (CD27+CD28+CD45RA+CCR7+) as compared to cells harvested from the 7-day process. Table 33 discloses the values from 4 individual samples per identified group.

TABLE 33
Cells from the 3-day process had greater percentages of Tscm
cells as compared to cells harvested the 7-day process

| | | Avg. % of live CD3 T-cells |
|---|---|---|
| | | (CD27 + CD28 + CD45RA + |
| Group | Sample | CCR7 + CD62L+) |
| 3-day negative | 1 | 54.7 |
| controls | 2 | 41 |
| (n = 4) | 3 | 49.1 |
| | 4 | 26.2 |
| 7-day negative | 1 | 25.9 |
| controls | 2 | 26.5 |
| (n = 4) | 3 | 9.93 |
| | 4 | 2.94 |
| 3-day cells | 1 | 27.5 |
| (n = 4) | 2 | 17.6 |
| | 3 | 26.7 |
| | 4 | 11.9 |
| 7-day cells | 1 | 3.98 |
| (n = 4) | 2 | 6.79 |
| | 3 | 3.94 |
| | 4 | 0.77 |

Finally, as shown in Table 34, harvested cells generated from the 3-day process had lower percentages of terminally differentiated T-cells (CD45RA+CCR7−CD27−CD28−) as compared to cells harvested from the 7-day process. Table 34 discloses the values from 4 individual samples per identified group.

TABLE 34
Cells from the 3-day process had lower percentages
of terminally differentiated T-cells as compared
to cells harvested from a 7-day process.

| | | Avg. % of live CD3 T-cells |
|---|---|---|
| | | (CD45RA + CCR7 − CD27 − |
| Group | Sample | CD28 − CD62L−) |
| 3-day negative | 1 | 0.068 |
| controls | 2 | 0.17 |
| (n = 4) | 3 | 0.5 |
| | 4 | 0.32 |
| 7-day negative | 1 | 0.41 |
| controls | 2 | 0.33 |
| (n = 4) | 3 | 3.13 |
| | 4 | 0.28 |
| 3-day cells | 1 | 0.28 |
| (n = 4) | 2 | 0.49 |
| | 3 | 1.19 |
| | 4 | 0.58 |
| 7-day cells | 1 | 0.73 |
| (n = 4) | 2 | 0.29 |
| | 3 | 4.08 |
| | 4 | 0.32 |

Phenotypic and functional characteristics of anti-CD19/CD20 CAR T cells produced by a process having a lentivirus transduction step on day 1 or day 2 were compared. In both processes, T cell selection and activation occurred on day 0. CAR expression was measured by detecting an anti-CD19 CAR antibody. Table 35 shows CAR expression of transduced T cells on day 1 and day 2 in cells harvested on day 3 to day 8.

TABLE 35
Percent of CD3 T cells with CAR+ expression

| | Transduction at Day 1 | Transduction at Day 2 |
|---|---|---|
| Day 3 harvest | 57.7 | 48.6 |
| Day 4 harvest | 63.2 | 48.3 |
| Day 6 harvest | 80.3 | 53.1 |
| Day 8 harvest | 77.3 | 45.2 |

T cell subpopulation of CD27+CD28+CD45RA+CCR7+CD62L+ was measured for anti-CD19/CD20 CAR T cells transduced on day 1 and day 2. Table 36 shows the percentage of juvenile CAR+ T cells produced by each method.

TABLE 36
Percent of CD27 + CD28 + CD45RA + CCR7 +
CD62L+ in CAR+ Cells

| | Transduction at Day 1 | Transduction at Day 2 |
|---|---|---|
| Day 0 harvest | 52.5 | 52.5 |
| Day 3 harvest | 46.9 | 43.6 |
| Day 4 harvest | 24.9 | 23.8 |
| Day 6 harvest | 14.4 | 22.5 |
| Day 8 harvest | 15.6 | 23.8 |

Day 0 samples in Table 36 were total T cells.

Functional assays of cytotoxicity were compared in anti-CD19/CD20 CAR T cells transduced on day 1 (T-D1) or day 2 (T-D2). Table 37 shows the results of 24-hour cytotoxicity assays having an E:T of 1:3 in three target cell lines (Nalm 6 WT, Raji WT, and Nalm 6 CD19 KO) on harvest days 3, 4, 6, and 8 (H-D3, H-D4, H-D6, and H-D8, respectively).

TABLE 37
24 hour cytotoxicity assay (% of killing), E:T = 1:3

| | | |
|---|---|---|
| Nalm6 WT | Raji WT | Nalm6 CD19 KO |

| | T-D1 | T-D2 | T-D1 | T-D2 | T-D1 | T-D2 |
|---|---|---|---|---|---|---|
| H-D3 | 72.9 | 70.0 | 41.6 | 41.2 | 12.6 | 6.9 |
| H-D4 | 65.9 | 60.9 | 36.3 | 29.2 | 2.9 | −3.5 |
| H-D6 | 75.7 | 72.0 | 48.4 | 49.6 | −9.0 | −11.0 |
| H-D8 | 87.3 | 77.5 | 61.3 | 48.4 | 1.6 | −7.7 |

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A method for preparing transduced lymphocytes, comprising
incubating a sample comprising lymphocytes, obtained from a donor subject, with a polynucleotide vector to transduce the lymphocytes to produce transduced lymphocytes; and
culturing the sample comprising the transduced lymphocytes for less than 72 hours before the lymphocytes are harvested to produce a harvested sample.

2. (canceled)

3. (canceled)

4. The method of claim 1, wherein the incubation is carried out in a closed system, and wherein the closed system has an inner surface area of at least 1500 cm².

5. (canceled)

6. The method of claim 4, wherein the closed system has an inner surface coated with a recombinant human fibronectin, wherein the coating is carried out with a solution that comprises about 1-10 μg/ml of the recombinant human fibronectin.

7. (canceled)

8. (canceled)

9. The method of claim 4, wherein the sample in the closed system comprises at least 1.5×10⁸ lymphocytes.

10. (canceled)

11. (canceled)

12. The method of claim 1, wherein the harvested sample comprises CD3+ cells, CD4+ T cells, and CD8+ T cells.

13. (canceled)

14. The method of claim 12, wherein at least 20% of the CD4+ T cells are naïve T cells, and no more than 12% of the CD4+ T cells are effector memory T cells.

15. (canceled)

16. The method of claim 12, wherein at least 10% of the CD8+ T cells are naïve T cells, and no more than 30% of the CD8+ T cells are effector memory T cells.

17. (canceled)

18. (canceled)

19. (canceled)

20. The method of claim 12, wherein at least 80% of the CD4+ T cells are CCR7+ cells, and wherein at most 20% of the CD4+ T cells are a combination of effector memory T cells and effector T cells.

21. The method of claim 12, wherein at least 60% of the CD8+ T cells are CCR7+ cells, and wherein at most 40% of the CD8+ T cells are a combination of effector memory T cells and effector T cells.

22. (canceled)

23. (canceled)

24. (canceled)

25. The method of claim 1, further comprising contacting the sample with a lymphocyte stimulating agent to activate the lymphocytes.

26. The method of claim 25, wherein the activating the sample is prior to incubating the sample with the polynucleotide vector.

27. (canceled)

28. The method of claim 25, wherein the activating the sample is after the sample is incubated with the polynucleotide vector.

29. (canceled)

30. (canceled)

31. (canceled)

32. The method of claim 30 or 31, wherein a total of 10,000 to 1,000,000 harvested lymphocytes per kilogram of the subject are administered to the subject.

33. (canceled)

34. (canceled)

35. (canceled)

36. (canceled)

37. The method of claim 1, wherein the polynucleotide vector encodes a chimeric antigen receptor (CAR) or a T cell receptor (TCR).

38. The method of claim 37, wherein the CAR comprises an intracellular costimulatory domain, and wherein the intracellular costimulatory domain is a signaling region of CD28.

39. (canceled)

40. (canceled)

41. The method of claim 38, wherein the CAR recognizes a tumor antigen, and wherein the tumor antigen is CD19, CD20, and/or CLL-1.

42. (canceled)

43. (canceled)

44. A population of cells prepared from a blood sample of a donor subject, comprising CD4+ T cells and CD8+ T cells, wherein:
at least 20% of the CD4+ T cells are naïve T cells, and no more than 12% of the CD4+ T cells are effector memory T cells;
at least 10% of the CD8+ T cells are naïve T cells, and no more than 30% of the CD8+ T cells are effector memory T cells;
at least 50% of the cells are CD3+ T cells; and
at least 25% of all T cells are transduced with a polynucleotide vector encoding a CAR or TCR.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A population of cells prepared from a blood sample of a donor subject, comprising CD4+ T cells and CD8+ T cells, wherein:
at least 80% of the CD4+ T cells are CCR7+ cells; at least 60% of the CD8+ T cells are CCR7+ cells;
at most 20% of the CD4+ T cells are a combination of effector memory T cells and effector T cells; and
at most 40% of the CD8+ T cells are a combination of effector memory T cells and effector T cells.

59. A pharmaceutical composition comprising the population of cells of claim 48.

60. A method for administering T cells to a subject, comprising injecting to the subject a harvested sample prepared by the method of claim 1.

61. (canceled)

62. (canceled)

63. (canceled)
